SEQUENCE LISTING (47 sequences)

SEQ ID NO: 1 - DNA, 19 bp, Artificial Sequence
  primer HsgA1
aaggtgtcaatyatgtttg

SEQ ID NO: 2 - DNA, 14 bp, Artificial Sequence
  primer HsgA2
acggttacttkttt

SEQ ID NO: 3 - DNA, 18 bp, Artificial Sequence
  primer HsgB1
tctattccctacctggat

SEQ ID NO: 4 - DNA, 17 bp, Artificial Sequence
  primer HsgB2
actcttaacggcagtag

SEQ ID NO: 5 - DNA, 32 bp, Artificial Sequence
  ADE1Bgl2S
gaagatctttatagatggaatggtgccaacat

SEQ ID NO: 6 - DNA, 31 bp, Artificial Sequence
  primer ADE1Hi3AS
cccaagcttaaaactcttctcgctggcagtc

SEQ ID NO: 7 - DNA, 17 bp, Artificial Sequence
  primer HsgC1
acctttgactcttctgt

SEQ ID NO: 8 - DNA, 17 bp, Artificial Sequence
  primer HsgC2
tccttgtatttagtatc

SEQ ID NO: 9 - DNA, 18 bp, Artificial Sequence
  primer HsgD1
ccatcatgttcgactcct

SEQ ID NO: 10 - DNA, 17 bp, Artificial Sequence
  primer HsgD2
aggtagccggtgaagcc

SEQ ID NO: 11 - DNA, 18 bp, Artificial Sequence
  HsgE1
gactcttccgtcagctgg

SEQ ID NO: 12 - DNA, 17 bp, Artificial Sequence
  primer HsgE2
gctggtaacggcgctct

SEQ ID NO: 13 - DNA, 17 bp, Artificial Sequence
  primer HsgF1
atttctattccttcgcg

SEQ ID NO: 14 - DNA, 17 bp, Artificial Sequence
  primer HsgF2
tcaggcttggtacggcc

SEQ ID NO: 15 - DNA, 430 bp, Adenovirus
  misc_feature: isolate X765551Ko
ggcaccttttaccttaaccacactttcaagaaggtctccatcatgtttgactcctcagtcagctggcctggcaatgacaggctgttgagcccaaatgagtttgaaatcaagcgcactgtggacggggaagggtacaatgtggcccaatgtaacatgaccaaagactggttcctggttcagatgcttgccaactacaacattggctaccagggcttttacatccctgagggatacaaggatcgcatgtactcctttttcagaaacttccagcctatgagcaggcaggtggttgatgaggttaattacactgactacaaagccgtcaccttaccataccaacacaacaactctggctttgtagggtatcttgcacctactatgagacaaggggaaccttacccagccaattatccatacccgctcatcggaa

SEQ ID NO: 16 - DNA, 430 bp, Adenovirus; identical to SEQ ID NO: 15
  misc_feature: isolate M2-3s

SEQ ID NO: 17 - DNA, 430 bp, Adenovirus
  misc_feature: isolate M7-1s
ggcaccttctaccttaaccacactttcaagaaggtctccatcatgtttgactcctcagtcagctggcctggcaatgacaggctgttgagcccaaatgagtttgaaatcaagcgcactgtggacggggaaggatacaacgtggcacaatgcaacatgaccaaagactggttcctagttcagatgcttgccaactacaacattggctaccagggcttttacatccctgagggatacaaggatcgcatgtactcttttttcagaaacttccagcctatgagcaggcaggtggttgatgaggttaattacactgactacaaagccgtcaccttaccataccaacacaacaactctggctttgtagggtaccttgcacctactatgagacaaggggaaccttacccagccaattatccatacccgctcatcggaa

SEQ ID NO: 18 - DNA, 430 bp, Adenovirus
  misc_feature: isolate M8-2s
ggcaccttctaccttaaccacactttcaagaaggtctccatcatgtttgactcctcagtcagctggcctggcaatgacaggctgttgagcccaaatgagtttgaaatcaagcgcactgtggacggggaaggatacaacgtggcacaatgcaacatgaccaaagactggttcctagttcagatgcttgccaactacaacattggctaccagggcttttacatccctgagggatacaaggatcgcatgtactcttttttcagaaacttccagcctacgagcaggcaggtggttgatgaggttaattacactgactacaaagccgtcaccttaccataccaacacaacaactctggctttgtagggtaccttgcacctactatgagacaaggggaaccttacccggccaattatccatacccgctcatcggaa

SEQ ID NO: 19 - protein, 143 aa, Adenovirus isolate X765551Ko
GTFYLNHTFKKVSIMFDSSVSWPGNDRLLSPNEFEIKRTVDGEGYNVAQCNMTKDWFLVQMLANYNIGYQGFYIPEGYKDRMYSFFRNFQPMSRQVVDEVNYTDYKAVTLPYKHNNSGFVGYLAPTMRQGEPYPANYPYPLIG

SEQ ID NO: 20 - protein, 143 aa, Adenovirus isolate M2-3s; identical to SEQ ID NO: 19

SEQ ID NO: 21 - protein, 143 aa, Adenovirus isolate M7-1s; identical to SEQ ID NO: 19

SEQ ID NO: 22 - protein, 143 aa, Adenovirus isolate M8-2s
GTFYLNHTFKKVSIMFDSSVSWPGNDRLLSPNEFEIKRTVDGEGYNVAQCNMTKDWFLVQMLANYNIGYQGFYIPEGYKDRMYSFFRNFQPTSRQVVDEVNYTDYKAVTLPYKHNNSGFVGYLAPTMRQGEPYPANYPYPLIG

SEQ ID NO: 23 - DNA, 430 bp, Adenovirus
  misc_feature: isolate AF065068Ko
ggcactttttaccttaaccacactttcaagaaggtctccatcatgtttgactcctcagtcagctggcctggcaatgacaggctgttgtctccaaatgagtttgaaatcaagcgcactgtggatggggaaggatacaatgtggcccaatgcaacatgaccaaagactggttcctggttcagatgcttgccaactacaacattggctaccagggcttttacatccctgagggatacaaggatcgcatgtactcctttttcagaaacttccagcctatgagcaggcaggtggttgatgaggttaattacactgactacaaagccgtcaccttaccatatcaacacaacaactctggctttgtaggataccttgcgcctactatgagacaaggggaaccttacccagccaattatccatacccgctcatcggaa

SEQ ID NO: 24 - DNA, 430 bp, Adenovirus
  misc_feature: isolate M6-1s
ggcaccttttaccttaaccacactttcaagaaggtctccatcatgtttgactcctcagtcagctggcctggcaatgacaggctgttgtctccaaatgagtttgaaatcaagcgcactgtggatggggaaggatacaatgtggcccaatgcaacatgaccaaagactggttcctggttcagatgcttgccaactacaacattggctaccagggcttttacatccctgagggatacaaggatcgcatgtactcctttttcagaaacttccagcctatgagcaggcaggtggttgatgaggttaattacactgactacaaagccgtcaccttaccatatcaacacaacaactctggctttgtaggataccttgcgcctactatgagacaaggggaaccttacccagccaattatccatacccgctcatcggaa

SEQ ID NO: 25 - protein, 143 aa, Adenovirus isolate AF065068Ko
GTFYLNHTFKKVSIMFDSSVSWPGNDRLLSPNEFEIKRTVDGEGYNVAQCNMTKDWFLVQMLANYNIGYKGFYIPEGYKDRMYSFFRNFQPMSRQVVDEVNYTDYKAVTLPYKHNNSGFVGYLAPTMRQGEPYPANYPYPLIG

SEQ ID NO: 26 - protein, 143 aa, Adenovirus isolate M6-1s; identical to SEQ ID NO: 25

SEQ ID NO: 27 - DNA, 430 bp, Adenovirus; identical to SEQ ID NO: 15
  misc_feature: isolate AF065065Ko

SEQ ID NO: 28 - DNA, 430 bp, Adenovirus
  misc_feature: isolate M3.3P-2
ggcaccttttaccttaaccacactttcaagaaggtctccatcatgtttgactcctcagtcagctggcctggcaatgacaggctgttgagcccaaatgagtttgaaatcaagcgcactgtggacggggaagggtacaatgtggcccanngtaacatgaccaaagactggttcctggttcagatgcttgccaactacaacattggctaccagggcttttacatncctgagggatacaaggatcgcatgtactcctttttcagaaacttccagcctatgagcaggcaggtggctgatgaggntaattacactgactacaaagccggcaccttaccataccaacacaacaactctggctttgtagggtatcttgcacctactatgagacaaggggaaccttacccagccaattatccatacccgctcatcggaa

SEQ ID NO: 29 - DNA, 430 bp, Adenovirus; identical to SEQ ID NO: 15
  misc_feature: isolate M5-1s

SEQ ID NO: 30 - DNA, 430 bp, Adenovirus; identical to SEQ ID NO: 17
  misc_feature: isolate M9-2s

SEQ ID NO: 31 - protein, 143 aa, Adenovirus isolate AF065065Ko
GTFYLNHTFKKVSIMFDSSVSWPGNDRLLSPNEFEIKRTVDGEGYNVAQCNMTKDWFLVQMLANYNIGYQGFYIPEGYKDRMYSFFRNFQPMSRQVVDEVNYTDYKAVTLPYQHNNSGFVGYLAPTMRQGEPYPANYPYPLIG

SEQ ID NO: 32 - protein, 143 aa, Adenovirus isolate M3-3p
  MISC_FEATURE (49)..(49): Xaa is any amino acid
GTFYLNHTFKKVSIMFDSSVSWPGNDRLLSPNEFEIKRTVDGEGYNVAXXNMTKDWFLVQMLANYNIGYQGFYXPEGYKDRMYSFFRNFQPMSRQVADEXNYTDYKAGTLPYQHNNSGFVGYLAPTMRQGEPYPANYPYPLIG

SEQ ID NO: 33 - protein, 143 aa, Adenovirus isolate M5-1s; identical to SEQ ID NO: 31

SEQ ID NO: 34 - protein, 143 aa, Adenovirus isolate M9-2s; identical to SEQ ID NO: 31

SEQ ID NO: 35 - DNA, 430 bp, Adenovirus; identical to SEQ ID NO: 15
  misc_feature: isolate M2-3s

SEQ ID NO: 36 - DNA, 430 bp, Adenovirus; identical to SEQ ID NO: 15
  misc_feature: isolate M5-1s

SEQ ID NO: 37 - DNA, 430 bp, Adenovirus; identical to SEQ ID NO: 24
  misc_feature: isolate M6-1s

SEQ ID NO: 38 - DNA, 430 bp, Adenovirus; identical to SEQ ID NO: 17
  misc_feature: isolate M7-1s

SEQ ID NO: 39 - DNA, 430 bp, Adenovirus; identical to SEQ ID NO: 18
  misc_feature: isolate M8-2s

SEQ ID NO: 40 - DNA, 430 bp, Adenovirus; identical to SEQ ID NO: 17
  misc_feature: isolate M9-2s

SEQ ID NO: 41 - protein, 143 aa, Adenovirus isolate M2-3s; identical to SEQ ID NO: 19

SEQ ID NO: 42 - protein, 143 aa, Adenovirus isolate M5-1s; identical to SEQ ID NO: 19

SEQ ID NO: 43 - protein, 143 aa, Adenovirus isolate M6-1s; identical to SEQ ID NO: 19

SEQ ID NO: 44 - protein, 143 aa, Adenovirus isolate M7-1s; identical to SEQ ID NO: 19

SEQ ID NO: 45 - protein, 143 aa, Adenovirus isolate M8-2s; identical to SEQ ID NO: 22

SEQ ID NO: 46 - protein, 143 aa, Adenovirus isolate M9-2s; identical to SEQ ID NO: 19

SEQ ID NO: 47 - DNA, 720 bp, Adenovirus
  misc_feature: promoter sequence of the adenoviral protein\ E1A (as shown in fig. 5)
ctctctatataatataccttatagatggaatggtgccaacatgtaaatgaggtaatttaaaaaagtgcgcgctgtgtggtgattggctgtggggtgaatgactaacatgggcggggcggccgtgggaaaatgacgtgacttatgtgggaggagttatgttgcaagttattgcggtaaatgtgacgtaaaaggaggtgtggtttgaacacggaagtagacagttttcccacgcttactggtaggatatgaggtagttttgggcggatgcaagtgaaaattctccattttcgcgcgaaaactgaatgaggaagtgaatttctgagtcatttcgcggttatgacagggtggagtatttgccgagggccgagtagactttgaccgtttacgtggaggtttcgattaccgtgtttttcacctaaatttccgcgtacggtgtcaaagtcctgtgtttttacgtaggtgtcagctgatcgctagggtatttaaacctgacgagttccgtcaagaggccactcttgagtgccagcgagaagagttttctcctccgcgccgcaagtcagttctgcgctttgaaaatgagacacctgcgcttcctgccacaggagattatctccagtgagaccgggatcgaaatactggagtttgtggtaaataccctaatgggagacgacccggaaccgccagtgcagccttttgatccacctacgctgcacgatctgt

(19) United States
(12) Patent Application Publication
(10) Pub. No.: US 2009/0304708 A1
(43) Pub. Date: Dec. 10, 2009

(54) Preparation for the Prevention and/or Treatment of a Tissue Change of Mesenchymal Origin

(76) Inventor: Jorn Bullerdick, Bremen (DE)

Correspondence Address: ROCKEY, MILNAMOW & KATZ, LTD., TWO PRUDENTIAL PLAZA, STE. 4700, 180 NORTH STETSON AVENUE, CHICAGO, IL, 60601, US

(21) Appl. No.: 12/404,292

(22) Filed: Mar. 14, 2009

Related U.S. Application Data
(63) Continuation of application No. 11/601,255, filed on Nov. 17, 2006, now abandoned.
Application No. 11/601,255 is a continuation of application No. 09/890,684, filed on Aug. 3, 2001, now abandoned.

(30) Foreign Application Priority Data
Feb. 4, 1999 (DE) 199 04 514.3
Sep. 13, 1999 (DE) 199 43 786.6
Sep. 13, 1999 (DE) 199 43 787.4

(51) Int. Cl. A61K 39/42; C12Q 1/68; C40B 30/04; G01N 33/53; C12Q 1/70; A61P 37/04

(52) U.S. Cl. 424/147.1; 424/159.1; 435/6; 506/9; 435/7.1; 435/5

(57) ABSTRACT

The present invention concerns a preparation for preventing and/or treating a tissue change, wherein the tissue change involves tissue of mesenchymal origin or tissue changes derived therefrom and the preparation contains an antiviral agent.

The invention concerns preparations for preventing and/or treating a tissue change, the tissue change involving tissue of mesenchymal origin, use of the preparation, methods for producing a preparation for preventing and/or treating tissue changes of mesenchymal origin and/or to determine viruses against which the preparation according to the invention is directed and uses of the method, a device for determining viruses involved in the pathogenesis of tissue changes of mesenchymal origin and methods for diagnosing a tissue change, the tissue change being a change in tissue of mesenchymal origin.

The treatment of tissue changes such as tumours and carcinomas is still comparatively limited to chemotherapy and its diverse modifications and surgical interventions despite the concentrated efforts of modern medicine. It is far more difficult to prevent such tissue diseases. The usual approach is to exclude the factors responsible for the tissue change from the individual sphere of life which, however, is not always possible.

A particular problem area is to prevent and treat tissue changes of mesenchymal origin. The aetiology underlying such diverse tumours as leiomyoma, endometrial polyp, endometriosis, hamartoma of the lung and the mamma, prostate adenoma, atheroma and many more is still unknown today which consequently only leaves a symptomatic treatment for this type of disease. Prevention is especially difficult under these circumstances since an individual does not know how his behaviour can reduce the risk of contracting such a tissue change.

Reference is made to leiomyomas of the uterus as an example. These are a problem in health politics although they are a benign growth. Thus studies have shown that in West European countries every third woman has uterine leiomyomas and that for example in the USA every fifth visit to a gynaecologist is due to myomas (Morton, C. C.; Am. J. Pathol. 1998; 153(4): 1050-20). When symptoms of these leiomyomas become manifest, this can be for example associated with considerable bleeding and thus to health risks, pain and incontinence of urine. In addition these leiomyomas can also result in infertility in the affected women.

Despite diverse efforts the aetiology/pathogenesis of uterine leiomyomas has previously been unclear (Morton supra). Various potential causes of uterine leiomyomas are discussed for example by Cramer, S. F. et al. (Cramer S. F.; J. Reprod. Med. 1995, 40(8): 595-600) such as for example damage and repair of the endometrium. The involvement of oestrogen and/or progesterone and oestrogen and/or progesterone receptors in the formation of neoplasms of the smooth muscle of the uterus has also been discussed in the literature e.g. by Tiltman A. J. (Tiltman A. J.; Curr. Opin. Obstet. Gynecol., 1997; 9(1): 48-51). However, as a result of various investigations the concept has developed that mutations of genes of the HMGI(Y) family are involved (see Schoenmakers E. F. et al.; Nat. Genet. 1995, 10(4): 436-44) although it remains to be clarified whether the said mutations are primary or secondary events.

Despite this finding only two methods of treatment are currently available for myomas i.e. the operative removal of the uterus (hysterectomy), thus there are ca. 200,000 hysterectomies per year in the USA alone (Morton, supra) or myoma enucleation i.e. the scraping-out the tumours while retaining the uterus. In the case of myoma enucleations it is possible to pre-operatively reduce the size of the myoma by medication with hormone antagonists but this is associated with undesired side effects since it can trigger menopausal symptoms.

Hence there is an urgent need for new concepts for the treatment and prevention of leiomyomas, especially those of the uterus, and endometrial polyps and endometriosis and suitable preparations for this.

Hence an object of the present invention is to provide preparations for preventing and treating tissue changes, the tissue change involving tissue of mesenchymal origin.

A further object of the present invention is to provide a method which allows those essential components to be determined which are suitable or necessary for the production of the preparation according to the invention.

Finally a further object of the invention is to provide a device for determining an agent involved in the pathogenesis of tissue changes, the tissue change involving tissue of mesenchymal origin.

Furthermore it is an object of the invention to provide a method for diagnosing a tissue change, the said tissue change involving tissue of mesenchymal origin, and a suitable kit therefor.

The object is generally achieved by a preparation for the prevention and/or treatment of a tissue change, wherein the tissue change involves tissue of mesenchymal origin or tissue changes derived therefrom and the preparation contains an antiviral agent.

The object is achieved according to the invention especially in a first aspect by a preparation for preventing and/or treating a tissue change wherein the tissue change involves at least one tissue of mesenchymal origin and the preparation contains an antiviral agent that is effective against a virus whose nucleic acid contains at least one binding site for a gene product of genes of the HMGI(Y) family or derivatives thereof.

In a second aspect the object is achieved according to the invention by a preparation for preventing and/or treating a tissue change wherein the tissue change involves at least one tissue of mesenchymal origin and the preparation contains an antiviral agent which is effective against a virus whose nucleic acid codes for a gene product wherein this gene product interacts with at least one gene product of genes of the HMGI(Y) family or derivatives thereof.

In the preparations according to the invention the binding site on the nucleic acid of the virus can have the structural and sequence features of a first AT-rich sequence.

In one embodiment the binding site on the nucleic acid of the virus can in addition to the first sequence have the following structural and sequence features such that
a second AT-rich sequence is present and
the first and second sequence are arranged at a spatial distance from one another.

In a preferred embodiment the spatial distance is selected such that the first sequence and the second sequence are arranged relative to one another in one plane on the nucleic acid.

With regard to the preparation according to the invention provision can additionally be made for the genes of the HMGI(Y) family to include MAG genes, HMGIC, HMGIY, aberrant transcripts of genes of the HMGI(Y) family and derivatives thereof.

In one embodiment the tissue of mesenchymal origin is at least partially infected with a virus.

In a preferred embodiment the virus is one which is described herein in particular in connection with the preparations according to the invention and the virus infecting the tissue can be one of them, and in preferred embodiments the virus is the one against which the preparation and/or antiviral agent according to the invention is effective.

In a further embodiment the tissue change involves tissue as its sole or obligatory component wherein at least some of the cells composing this tissue are infected with one of the viruses described herein.

Furthermore the tissue change can comprise a proliferation of at least one mesenchymal cell which is infected with the viruses described herein.

In a preferred embodiment the proliferation is a clonal proliferation.

With regard to the preparations according to the invention, the tissue proliferation can also include an epithelial component.

In a preferred embodiment the epithelial component has at least one cell which is infected with one of the viruses described herein.

Furthermore the cell infected with one of the viruses described herein can have a chromosomal change.

In this connection it is preferred that the chromosomal change affects at least one HMGI(Y) gene of the infected cell.

In a further preferred embodiment the HMGI(Y) gene is selected from the group comprising the MAG genes, HMGIC, HMGIY, aberrant transcripts of genes of the HMGI(Y) family and derivatives thereof.

With the preparations according to the invention the tissue change can be selected from the group comprising leiomyomas, in particular leiomyomas of the uterus; endometrial polyps, endometriosis, fibroadenomas, in particular fibroadenomas of the mamma; phyllodes tumours, in particular of the mamma; hamartomas, in particular of the mamma; prostate adenoma; lipomas; aggressive angiomyxomas; enchondromas; pleomorphic adenomas, especially of the salivary glands of the head; colon polyps, especially colon adenomas; hamartomas, especially of the lung; atheromas and carcinomas that develop therefrom.

In this connection the carcinomas that develop are selected in one embodiment from the group comprising colon carcinomas and prostate carcinomas.

In a further embodiment of the preparation according to the invention the virus is selected from the group comprising DNA viruses and in particular adenoviruses and herpes viruses.

In the preparations according to the invention the agent can be selected from the group comprising vaccines, antibodies, agents which inhibit the replication, transcription or translation of viral genes especially genes of adenoviruses and/or herpes viruses; agents which recognize and/or destroy cells infected with viruses and especially adenoviruses and/or herpes viruses; and agents which achieve an antiviral action by their effector cell stimulating action.

In a preferred embodiment the vaccine comprises an antibody which is directed against a virus as described herein or a part thereof.

In an alternative preferred embodiment the vaccine comprises a particle of a virus as described herein or a part thereof.

In a further embodiment of the preparations according to the invention the antibody is selected from the group comprising monoclonal antibodies, polyclonal antibodies, polyvalent antibodies, antibody fragments and derivatives thereof.

In a third aspect the object is achieved by the use of the preparation according to the invention to immunize against viruses that are associated with the pathogenesis and/or aetiology of the tissue changes as described herein.

In a fourth aspect the object is achieved by an application of the preparation according to the invention to produce a pharmaceutical composition comprising the preparation as claimed in one of the previous claims and a pharmaceutically acceptable carrier to prevent and/or treat the tissue changes as claimed in one of the previous claims or to immunize against viruses that are associated with the pathogenesis and/or aetiology of the tissue changes as claimed in one of the previous claims.

In this connection one embodiment provides that the immunization is an active immunization.

In a fifth aspect the object is achieved by a method for determining viruses that are suitable for producing a preparation for preventing and/or treating tissue changes as described herein and/or determining viruses against which the preparation according to the invention is directed, which comprises the steps:
a) Transfecting a cell culture having a normal karyotype which is derived from a tissue that contains the tissue change as claimed in one of the previous claims, with an expression vector for a gene of the HMGI(Y) family or a derivative thereof,
b) comparing the RNA pattern of the transfected cells with that of control cultures, and
c) examining RNA(s) that are expressed or expressed more strongly in the transfected cultures compared to the control cultures for the presence of viral elements by sequence homology.

In a sixth aspect the object is achieved by a method for determining viruses that are suitable for producing a preparation for preventing and/or treating tissue changes as described herein and/or determining viruses against which the preparation according to the invention is directed which comprises carrying out a PCR test wherein the primer (pairs) used for the PCR correspond to a viral nucleic acid sequence.

In a seventh aspect the object is achieved by a method for determining viruses that are suitable for producing a preparation for preventing and/or treating tissue changes as described herein and/or determining viruses against which the preparation according to the invention is directed, which comprises the steps:
a) Setting up a CDNA library of a tissue which contains the tissue change as claimed in one of the previous claims in which a gene of the HMGI(Y) family or a derivative thereof is activated or can be activated and
b) screening the CDNA library with a virus-specific probe or
c) analysing the cDNA clones for viral sequences or
d) comparing with a cDNA library from a normal reference tissue.

In one embodiment of the method according to the invention the gene of the HMGI(Y) family can be selected from the group comprising HMGIC, HMGIY, MAG, aberrant transcripts of genes of the HMGI(Y) family and derivatives thereof.

In a further embodiment the virus, the viral element or the virus-specific probe can be selected from the group comprising viruses as described herein i.e. those viruses whose nucleic acid contains at least one binding site for a gene product of genes of the HMGI(Y) family or derivatives thereof or viruses whose nucleic acid codes for a gene product and this gene product codes for at least one gene product wherein this gene product interacts with at least one gene product of genes of the HMGI(Y) family or derivatives thereof. The previously described viruses also include those which belong to the group of DNA viruses in particular adenoviruses, herpes viruses and papova viruses which have at least one of the two previously mentioned features.

In an eighth aspect the object is achieved by the use of one of the methods according to the invention to determine viruses against which an immunization can be carried out in order to prevent and/or treat tissue changes as described herein.

Finally in a ninth aspect the object is achieved by a device for determining a virus involved in the pathogenesis of tissue changes as described herein which contains a gene product of genes of the HMGI(Y) family or a part thereof or a derivative thereof which is bound to a carrier.

In one embodiment provision is made that the viral nucleic acid can in addition to the first sequence, also have the following structural and sequence features such that
a second AT-rich sequence is present and
the first and second sequence are arranged at a spatial distance from one another.

In a preferred embodiment the spatial distance is selected such that the first sequence and the second sequence are arranged relative to one another in one plane on the nucleic acid.

In a tenth aspect the object is achieved by a method for diagnosing a tissue change wherein the tissue change comprises a tissue change as described herein, in which a body fluid from a patient that may have such a tissue change is examined for the presence of antibodies against viruses as described herein.

In an eleventh aspect the object is achieved by a method for diagnosing a tissue change wherein the tissue change comprises a tissue change as described herein, in which a body fluid from a patient that may have such a tissue change is examined for the presence of antigens of viruses as described herein.

Furthermore in a twelfth aspect the object is achieved by a method for diagnosing a tissue change wherein the tissue change comprises a tissue change as described herein, in which a tissue sample is reacted with an agent that is selected from the group comprising antibodies which react with viruses as described herein and especially DNA viruses and quite especially adenoviruses and/or herpes viruses or parts thereof, antigens that are derived from viruses as described herein and in particular DNA viruses and especially adenoviruses and/or herpes viruses or parts thereof and nucleic acid which interacts with the nucleic acid of viruses as described herein and especially of DNA viruses and quite especially adenoviruses and/or herpes viruses, if viruses are present as described herein and especially DNA viruses and quite especially adenoviruses and/or herpes viruses, a complex is formed from the preparation and the virus, and the complex is detected.

The aspects one to twelve are also referred to in the following as “main aspect I”.

In a thirteenth aspect the object is achieved according to the invention by a preparation for preventing and/or treating a tissue change wherein the tissue change involves tissue of mesenchymal origin or tissue changes derived therefrom and the preparation contains an antiviral agent, the antiviral agent being effective against a virus from the group of DNA viruses and in particular adenoviruses and/or herpes viruses.

In a fourteenth aspect the object is achieved by a preparation for preventing and/or treating a tissue change wherein the tissue change involves tissue of mesenchymal origin or tissue changes derived therefrom and the tissue of mesenchymal origin is at least partially infected with a virus from the group of DNA viruses and in particular with adenoviruses and/or herpes viruses. This aspect can, however, also be an embodiment of the thirteenth aspect since the viruses are those that have also been described in connection with the preparation according to the invention in which the viruses are those against which the viral agent is active and/or which have infected the mesenchymal tissue and whose nucleic acid contains at least one binding site for a gene product of genes of the HMGI(Y) family or whose nucleic acid codes for a gene product which interacts with at least one gene product of genes of the HMGI(Y) family and hence, it is possible that the antiviral agent is effective against these viruses and/or that these viruses have infected the mesenchymal tissue. In other words the main aspect II provides that the virus is one as described herein and in particular a DNA virus and that this virus is the virus that has infected the mesenchymal tissue and/or is the one against which the inventive preparation and/or antiviral agent is effective.

In the case of the preparation according to the invention the agent can be selected from the group comprising vaccines, antibodies, agents which inhibit the replication, transcription or translation of viral genes especially genes of adenoviruses and/or herpes viruses; agents which recognize and/or destroy the cells infected with viruses and especially with adenoviruses and/or herpes viruses; and agents which achieve an antiviral action by their effector cell stimulating action.

In one embodiment of the preparation according to the invention the tissue change involves tissue as its sole or obligatory component wherein at least some of the cells which make up the tissue are infected with viruses and especially adenoviruses and/or herpes viruses.

In a further embodiment the tissue proliferation comprises a proliferation of at least one mesenchymal cell which is infected with viruses, especially adenoviruses and/or herpes viruses.

In particular this proliferation can be a clonal proliferation.

In one embodiment of the preparation according to the invention the tissue proliferation includes an epithelial component.

In particular this epithelial component has at least one cell which is infected with a virus from the group of DNA viruses, especially an adenovirus and/or herpes virus.

In a further preferred embodiment the cell infected with a virus from the group of DNA viruses, especially an adenovirus and/or herpes virus can have a chromosomal change.

In this connection it is preferred that the chromosomal change includes at least one HMGI(Y) gene of the infected cell.

In connection with the preparation according to the invention one embodiment provides that the tissue change is selected from the group comprising leiomyomas, in particular leiomyomas of the uterus; endometrial polyps, endometriosis, fibroadenomas, in particular fibroadenomas of the mamma; phyllodes tumours, in particular of the mamma; hamartomas, in particular of the mamma; prostate adenoma; lipomas; aggressive angiomyxomas; enchondromas; pleomorphic adenomas, especially of the salivary glands of the head; colon polyps, especially colon adenomas; hamartomas, especially of the lung; atheromas and carcinomas that develop therefrom.

In this connection the carcinomas that develop are selected from the group comprising colon carcinomas and prostate carcinomas in one particularly preferred embodiment.

In a preferred embodiment the vaccine is directed against a virus from the group of DNA viruses and in particular against an adenovirus and/or herpes virus.

In a particularly preferred embodiment the vaccine contains a virus particle or parts thereof.

In an alternative preferred embodiment the vaccine contains an antibody which is directed against the virus or a part thereof.

In a further embodiment the vaccine is directed against a virus whose nucleic acid contains at least one binding site for a gene product of genes of the HMGI(Y) family or derivatives thereof.

In an alternative further embodiment the vaccine is directed against a virus whose nucleic acid codes for at least one gene product, and this gene product interacts with at least one gene product of genes of the HMGI(Y) family or derivatives thereof.

In a preferred embodiment the binding site on the nucleic acid of the virus has the structural and sequence features of a first AT-rich sequence.

In this connection the binding site of the viral nucleic acid can, in addition to the first sequence, have the following structural and sequence features such that
a second AT-rich sequence is present and
the first and second sequence are arranged at a spatial distance from one another.

In this case the spatial distance is selected such that the first sequence and the second sequence are arranged relative to one another in one plane on the nucleic acid.

In the case of the preparation according to the invention the genes of the HMGI(Y) family can comprise MAG genes, HMGIC, HMGIY, aberrant transcripts of genes of the HMGI(Y) family and derivatives thereof.

In one embodiment of the preparation according to the invention the antibody is selected from the group comprising monoclonal antibodies, polyclonal antibodies, polyvalent antibodies, antibody fragments and derivatives thereof.

Furthermore the object of the invention is achieved in a fifteenth aspect by using the preparation according to the invention to immunize against viruses that are associated with the pathogenesis and/or aetiology of the tissue changes as described herein.

In a sixteenth aspect the object is achieved by an application of the preparation according to the invention to produce a pharmaceutical composition comprising the preparation according to the invention and a pharmaceutically acceptable carrier for the prevention and/or treatment of the tissue changes as described herein or for an immunization against viruses that are associated with the pathogenesis and/or aetiology of the tissue changes as described herein.

In this connection the immunization can be an active immunization.

In addition in a seventeenth aspect the object is achieved by a method for determining viruses that are suitable for producing a preparation according to the invention for preventing and/or treating tissue changes as described herein and/or for determining viruses against which the preparation is directed which comprises the steps:
a) Transfecting a cell culture having a normal karyotype which is derived from a tissue which contains the tissue change as claimed in one of the previous claims, with an expression vector for a gene of the HMGI(Y) family or a derivative thereof,
b) comparing the RNA pattern of the transfected cells with that of control cultures, and
c) examining RNA(s) that are expressed or expressed more strongly in the transfected cultures compared to the control cultures for the presence of viral elements by sequence homology.

Furthermore in an eighteenth aspect the object is achieved by a method for determining viruses that are suitable for producing a preparation according to the invention for preventing and/or treating tissue changes as described herein and/or for determining viruses against which the preparation as claimed in one of the previous claims is directed which comprises carrying out a PCR test wherein the primer (pairs) used for the PCR correspond to the viral nucleic acid sequence.

Furthermore in a nineteenth aspect the object is achieved by a method for determining viruses that are suitable for producing a preparation according to the invention for preventing and/or treating tissue changes as described herein and/or for determining viruses against which the preparation as claimed in one of the previous claims is directed which comprises the steps:
a) Setting up a CDNA library of a tissue which contains the tissue change as claimed in one of the previous claims in which a gene of the HMGI(Y) family or a derivative thereof is activated or can be activated and
b) screening the CDNA library with a virus-specific probe or
c) analysing the cDNA clones for viral sequences or
d) comparing with a cDNA library from a normal reference tissue.

In one embodiment of the method according to the invention the gene of the HMGI(Y) family is selected from the group comprising HMGIC, HMGIY, MAG, aberrant transcripts of the genes of the HMGI(Y) family and derivatives thereof.

In a further embodiment the virus, the viral element or the virus-specific probe can be selected from the group which comprises DNA viruses and especially adenoviruses and/or herpes viruses.

Finally in a twentieth aspect the object is achieved by the use of one of the methods according to the invention to determine viruses against which an immunization can be carried out in order to prevent and/or treat tissue changes as described herein.

In a twenty-first aspect the object is achieved by a device for determining a virus involved in the pathogenesis of tissue changes as described herein which contains a gene product of genes of the HMGI(Y) family or a part thereof or a derivative thereof which is bound to a carrier.

One embodiment of the device according to the invention provides that the viral nucleic acid can, in addition to the first sequence, have the following structural and sequence features such that
a second AT-rich sequence is present and
the first and second sequence are arranged at a spatial distance from one another.

In a further embodiment the spatial distance is selected such that the first sequence and the second sequence are arranged relative to one another in one plane on the nucleic acid.

In a twenty-second aspect the object is achieved by a method for diagnosing a tissue change wherein the tissue change comprises a tissue change as described herein, in which a body fluid is examined for the presence of antibodies against DNA viruses and especially adenoviruses and/or herpes viruses.

In a twenty-third aspect the object is achieved by a method for diagnosing a tissue change, wherein the tissue change comprises a tissue change as described herein, in which a body fluid is examined for the presence of antigens of DNA viruses and especially adenoviruses and/or herpes viruses.

Finally in a twenty-fourth aspect the object is achieved by a method for diagnosing a tissue change, wherein the tissue change comprises such a tissue change as described herein, in which a tissue sample is reacted with a preparation that is selected from the group comprising antibodies which react with DNA viruses and in particular adenoviruses and/or herpes viruses or parts thereof; antigens that are derived from DNA viruses and especially adenoviruses and/or herpes viruses or parts thereof and nucleic acid which interacts with the nucleic acid of DNA viruses and especially adenoviruses and/or herpes viruses, if DNA viruses and especially adenoviruses and/or herpes viruses are present, a complex is formed from the preparation and the DNA virus and in particular adenoviruses and/or herpes viruses and the complex is detected.

The aspects thirteen to twenty-four are referred to in the following as “main aspect II”.

Before the invention is elucidated in more detail in the following, the terminology will be defined to supplement the general understanding of a person skilled in this field.

Leiomyomas are in particular understood herein as benign tumours of the smooth musculature (definition according to Baltzer, J. et al.; “Gynäkologie—Ein kurzgefaβtes Lehrbuch”, 5^{th} edition, 1994, published by Thieme).

Endometrial polyps are herein understood in particular as hyperplasias and polypous growth forms of the endometrium having stromal and glandular (epithelial) or only stromal components.

Endometriosis is to be understood herein in particular as endometrial foci which are located at a site that is other than in the cavum uteri (definition according to Baltzer supra).

Myomas are understood in particular herein as leiomyomas (definition according to Baltzer supra).

The general basis for the invention is the surprising finding that tissue changes which involve a tissue that is of mesenchymal origin have a common pathogenicity mechanism. This pathogenicity mechanism relates especially to the main aspect I of the invention and is linked to the presence of a virus in the tissue of mesenchymal origin, the virus being one whose nucleic acid contains at least one binding site for a gene product of genes of the HMGI(Y) family or derivatives thereof or the virus being one whose nucleic acid codes for a gene product wherein this gene product interacts with at least one gene product of genes of the HMGI(Y) family or derivatives thereof. These viruses are also herein named “HMGI viruses” or referred to by the term “as described herein”. In other words this type of virus is a factor in the triggering of tissue changes which involve tissue of mesenchymal origin.

Especially with regard to the main aspect II, the invention is based on the surprising finding that tissue changes which involve a tissue of mesenchymal origin have a common pathogenicity mechanism and this mechanism is related to the presence of DNA viruses in the tissue of mesenchymal origin. In other words DNA viruses are a factor in triggering tissue changes which involve the tissue of mesenchymal origin.

In this connection within the group of DNA viruses the adenoviruses appear to be of particular importance for this. Another group of DNA viruses which are of particular importance for the tissue changes described herein which involve a tissue of mesenchymal origin are viruses of the herpes group which are referred to in the following and herein simply as herpes viruses. The DNA virus group which are of importance for the tissue changes described herein also includes the papova viruses.

Especially with regard to the main aspect II, the present invention is also based on the finding that different viruses and in particular DNA viruses or HMGI viruses can act synergistically effect with regard to the tissue changes described herein.

As a result of the findings described above it was possible for the inventor to create the preparations according to the invention. If viruses are causally involved in the tissue changes in one form or another, this causality and thus the development, maintenance and/or progression of the tissue changes can be influenced by antiviral agents. Thus, simply expressed, an antiviral agent can be used against tissue changes which are caused by or have been infected by a virus as described herein i.e. HMGI viruses and/or DNA viruses. Consequently this also means that those antiviral agents can be used for such tissue changes which are effective against the viruses described herein i.e. the viruses named herein as HMGI viruses and DNA viruses, in particular because and to the extent that they are directly or indirectly causally involved in the tissue change.

Thus the term “a virus as claimed in one of the previous claims” which is used especially in the claims refers back to the description of the viruses as given in one of the claims but without claiming the viruses per se in order not to repeat their features. In general this abbreviated notation refers to HMGI viruses and/or DNA viruses as described herein.

The tissue changes described herein can include tumours and/or carcinomas. With regard to the histological structure of the said tissue changes it can be stated that these can be composed completely of a tissue which has changed under the influence of HMGI viruses and/or DNA viruses (in particular adenoviruses and/or herpes viruses) which, provided the respective context gives no other meaning, are also referred herein as “the said viruses”, or a part of the tissue exhibiting the tissue change is composed of tissue that is under the influence of the said viruses and consequently this part of the tissue is only one component of the tissue change or tumour but nevertheless an obligatory component.

In addition the tissue change can be one in which the change is due to a proliferation of mesenchymal cells infected with the said viruses. A tissue change can at the same time also have an epithelial component. An epithelial component is understood herein as a part of the tissue change (for example of the tumour or the carcinoma) which can be ascribed to the epithelium with regard to its histological origin. This histological classification is based on the generally accepted criteria of histopathology.

With regard to the tissue changes a situation can also be present in which the proliferation is a clonal proliferation i.e. the proliferation originates from a single infection event in which a single cell is transformed as a result of infection with the said viruses. The transformation of this single cell changes the behaviour of the cell and in particular its state of differentiation and/or its growth behaviour which leads to a tissue change. If this tissue change originates from an individual cell infected with the said virus it is referred to as a monoclonal tumour, if the tissue change originates from several cells but only a few of which are infected with the said viruses it is referred to as an oligoclonal tumour. Both clonalities have their origin in the pathogenicity mechanism mediated by the said viruses which is disclosed herein.

The universal pathogenicity mechanism in which tissue that ultimately exhibits or forms the tissue change as result of infection of the mesenchymal cell components of tissues by the said viruses gives rise to a number of different potential infection scenarios. Thus for example in one case the primary infection can take place in and be limited to a tissue of mesenchymal origin. This can lead to further tissue changes in which epithilial proliferation occurs that is a direct or indirect consequence of the viral infection of the mesenchymal component. Examples of this are hamartomas and endometriosis. However, it is also possible for the primary infection to spread to other tissue components. In this process epithelial tissue may also be infected which is then a secondary infection and this secondarily infected epithelial tissue can also proliferate. It is also conceivable that epithelial tissue is firstly, i.e. primarily, infected by the said viruses and that this is a starting point for a secondary infection of the mesenchymal tissue or tissue component of the subsequent tissue change which then can lead to a proliferation of the mesenchymal tissue. An example of the latter scenario is adenoma of the colon.

The viruses described herein can be present in any form in the cell during or after the infection. Thus in the infected cells the virus can be present episomally or integrated into the host genome. It can also progress through a lytic cycle in which it is released into the environment and infects other cells as also described above. The virus can also be present in epithelial cells either episomally or integrated into the genome.

Adenovirus is understood herein quite generally as any member of the group of adenoviruses as described for example in Fields Virology 3^{rd} edition, Raven Publisher, Philadelphia, 1996 which has the properties described therein. The same applies to viruses from the group of herpes viruses i.e. herpes virus is understood herein quite generally as any virus from the group of herpes viruses e.g. also the cytomegalovirus as described in Fields Virology supra, which has the properties described therein. The term adenoviruses and/or herpes viruses also covers herein those viruses, especially in connection with the vaccine directed against adenoviruses and/or herpes viruses which have essential elements and/or properties of these viruses. These also include genetically modified viruses.

The group of DNA viruses also includes the polyoma viruses which also belong to the family of papovaviridae which also include the papilloma viruses and simian vacuolating virus 40 (SV 40). The family is characterized by an extreme thermal stability. Papoviridae are cubic DNA viruses without coats which have a diameter of 45 to 55 nm and comprise 72 capsomers and a cyclic double-stranded DNA. The statements made herein relating to or in connection with adenoviruses and herpes viruses also applies in the same sense to this group of DNA viruses.

As a result of this pathogenicity mechanism for tissue changes especially in humans which involve tissue of mesenchymal origin which according to the current opinion of the inventor is universal and is based on the involvement of the said viruses i.e. those which are described herein, it is possible to provide preparations and treatment plans for the therapy of all those tissue changes which involve tissue of mesenchymal origin. The same applies to the diagnosis of such tissue changes.

Moreover knowledge of this pathogenicity mechanism also allows a prevention using the preparation according to the invention. This fundamental finding can be used to provide antiviral agents for the treatment of such tissue changes. In this respect these agents can also contain antiviral agents that are already known provided they are suitable for influencing the activity of the viruses described herein i.e. HMGI viruses and/or DNA viruses. Potential points of attack for such an impairment of viral activity are conceivably all individual stages or partial aspects including the absorption process, internalisation process, integration of viral DNA into the host genome or stabilization in the cytoplasm of the host cell, replication, transcription and translation, assembly of the viral capsid and release of the viral capsid. Furthermore vaccines against these viruses are also especially suitable for the treatment and in particular prevention of such tissue changes as elucidated in more detail in the following.

Tissue changes are listed as examples in the following table 1 for which the agents according to the invention can be used or for which already known antiviral agents as shown for example in table 2 can be used for treatment and prevention.

TABLE 1

| Tissue | Mesenchymal | | Formation of |
|---|---|---|---|
| change/tumour | component | Epithelial component | malignant tumours |
| leiomyomas (uterus) | such as smooth | absent | potential for |
| | musculature, | | sarcomatous |
| | monoclonal ca. 15% | | transformation |
| | mutations of HMGI(Y) | | doubtful; if at all very |
| | genes | | rare |
| endometrial polyps | stroma, monoclonal, | polyclonal | carcinoma formation |
| | ca. 45% mutations of | | from the epithelial |
| | HMGI(Y) genes | | component |
| endometriosis | present (stroma) | present | is not discussed |
| fibroadenomas | stroma, clonal | glandular epithelium | potential for carcinoma |
| (mamma) | chromosomal changes | | formation from the |
| | have been described | | epithelial component |
| | ca. 10% mutations of | | doubtful; if at all very |
| | the HMGI(Y) genes | | rare |
| phyllodes tumours | stroma, clonal | glandular epithelium | partially malignant |
| (mamma) | chromosomal | | |
| | changes/mutations of | | |
| | the HMGI(Y) genes | | |
| | have been described | | |
| hamartomas (mamma) | such as fat tissue, | glandular epithelium | potential for carcinoma |
| | monoclonal mutations | | formation from the |
| | of the HMGI(Y) genes | | epithelial component |
| | have been described | | doubtful; if at all very |
| | | | rare |
| prostate adenomas | such as smooth | glandular epithelium | formation of prostate |
| | musculature (synonym: | | carcinomas |
| | adenomyomas), clonal | | |
| | chromosomal changes | | |
| | have been described | | |
| lipomas | such as fat tissue, | absent | almost no |
| | monoclonal, ca. 40% | | transformation into |
| | mutations of the | | liposarcoma |
| | HMGI(Y) genes | | |
| aggressive | myxoid, clonal | absent | characteristic: wide |
| angiomyxomas | chromosomal changes | | lumen vessels |
| | (target gene: HMGIC) | | |
| enchondromas | such as hyaline | absent | almost no |
| | cartilage, clonal | | transformation into a |
| | chromosomal changes | | chondrosarcoma |
| | have been described | | |
| | (a.o. 12q14-15) | | |
| pleomorphic adenomas | mesenchymal and | epithelial component, | formation of |
| (salivary glands of the | common clonal is | origin of both | carcinomas ex |
| head) | assumed | components ca. 20% | pleomorphic adenoma |
| | | HMGIC mutations | |
| colon polyps (colon | stroma | intestinal epithelium | adenoma carcinoma |
| adenomas) | | | sequence established |
| | | | histologically |
| hamartomas (lung) | such as hyaline | epithelial gaps | potential for carcinoma |
| | cartilage, fat tissue, | (bronchial epithelium) | formation from the |
| | smooth musculature, | | epithelial component |
| | monoclonal, ca. 70% | | doubtful; if at all very |
| | mutations of the | | rare |
| | HMGI(Y) genes | | |
| atheromas | including smooth | endothelial coating | is not discussed |
| | musculature, clonal | | |
| | chromosomal changes | | |
| | have been described | | |

TABLE 2
Review of various antiviral compounds which can be
used within the scope of the present invention.

| Active substance | Trade name (representative) | Mode of action |
|---|---|---|
| adenosine arabinoside | Vidarabin | inhibition of viral replication |
| bromovinyluridine arabinoside | Brovavir, Sorivudin | inhibition of viral replication |
| 9-(1,3-dihydroxy-2- | Gabciclovir | inhibition of viral polymerase |
| propoxy)methyl guanine | | |
| trisodium salt of phosphono- | Foscarnet | inhibition of viral polymerase |
| formic acid | | |
| interferon α | Roferon A | inhibition of viral protein |
| | | synthesis |
| interferon β | Fiblaferon | inhibition of viral protein |
| | | synthesis |
| interferon γ | Imukin | inhibition of viral protein |
| | | synthesis |
| immunoglobulin | Pentaglobin | antibody-mediated antiviral |
| | | action |

In addition to the universal pathogenicity mechanism described above, the inventor has also surprisingly found that the formation of the tissue changes which involve tissue of mesenchymal origin or of corresponding tumours that start with infection by the said viruses described above, is promoted in a synergistic manner by an additional event. This additional event is a chromosomal change and especially one affecting the HMGI(Y) genes, i.e. breakage points of structural chromosomal aberrations are located either within the genes or at such a distance from them that the structural chromosomal aberrations lead to a transcriptional de-regulation of the HMGI(Y) genes. This chromosomal change then results in the expression of modified cellular gene products which interact with certain viral sequences which is the basis for the observed synergistic effect in the genesis of the tissue changes. The term HMGI(Y) genes is further elucidated in the following in connection with the vaccines disclosed as preparations according to the invention.

Examples of tissue changes which, in addition to the infection of the mesenchymal tissue component, also exhibit a chromosomal change of HMGI(Y) are shown in table 1 above whereby reference is made by way of example to endometrial polyps, endometriotic foci, hamartomas of the lung, lipomas, fibroadenomas of the mamma and pleomorphic adenomas of the salivary glands. As also shown in particular in table 1 the proportion can vary of those tissue changes which, in addition to the infection by the said viruses i.e. HMGI viruses and/or DNA viruses, also carry a chromosomal change of the HMGI(Y) genes. The detection of the chromosomal change is described for example in Kazmierczak et al., Oncogene 12: 515-521.

As already mentioned above the prevention and treatment of mesenchymal tissue changes which involve tissue of mesenchymal origin starts with an antiviral treatment. However, the tissue changes can also include chromosomal changes affecting the HMGI(Y) genes in addition to the viral infection. Since such tissue changes are also ultimately due to a viral activity, they can be treated or prevented with the preparations proposed herein. This would also explain the mutations of the genes of the HMGI(Y) family in uterine leiomyomas investigated by Schoenmakers (supra) which are described above although it would not be possible to deduce the technical teaching disclosed herein from the finding described therein. The pathogenesis of leiomyomas is not only due to mutations, in particular chromosomal aberrations in the region of loci of members of the HMGI(Y) gene family, but, at least in some of these cases i.e. tissue changes involving a tissue of mesenchymal origin with a mutation in the region of the HMGI(Y) genes, it is a result of the interaction between mutation events of members of the HMGI(Y) family and a virus, in particular a transforming virus or infection with such a virus whereby the viral infection itself is already sufficient to trigger the formation of myomas the growth potential of which is increased by a—in some cases subsequent—mutation of genes of the HMGI(Y) family.

Without intending to be limited by these assumptions in the following and especially not in detail, it appears at present that, as a result of infection with a transforming virus and its subsequent persistence, presumably after integration into the cellular genome, the transforming viral proteins may in some cases be expressed only weakly in cells that do not have additional mutations of genes of the HMGI(Y) family and the resulting tumours therefore grow very slowly and remain small. Reactivation of genes of the HMGI(Y) family caused by a mutation e.g. by a shift of enhancers to a position next to the genes, increases the activation of the genes of the transforming proteins and overall their expression is elevated. The transforming proteins which are present in an increased amount in the cell lead to a considerably higher growth activity of the corresponding tumours and hence to an increased tumour growth.

Specifically one has found that the gene products of members of the genes of the HMGI(Y) family which can typically bind to nucleic acids as described for example by French, S. W. et al. (French, s. W. et al.; Mol. Cell Biol., 1966; 16(10): 5393-99; Yie, J. et al.; Mol. Cell Biol. 1997, 17(7): 3649-62), also bind to the nucleic acid of transforming viruses and if this binding occurs in the area of the regulatory regions of the nucleic acid (e.g. promoters and enhancer), the gene products of the genes of the HMGI(Y) family influence the transcription rate of the viral transforming proteins.

It has additionally been found that the gene products of the members of the genes of the HMGI(Y) family can also interact with a gene product of a viral nucleic acid.

This interaction can be a direct interaction of the two gene products i.e. between the gene product of the viral nucleic acid and that of the gene of the HMGI(Y) family. Another type of interaction can be mediated by another component i.e. there is no direct interaction between the two gene product species. This mediating other component can for example be a nucleic acid and in particular a nucleic acid which has a binding site for a gene product of genes of the HMGI(Y) family as described above.

As a result of this interaction between gene products of genes of the HMGI(Y) family and the nucleic acid of transforming viruses and in particular the regulatory regions of the nucleic acid (e.g. promoters and enhancer) of these viruses, it is possible to counteract the formation of tissue changes involving tissue of mesenchymal origin as listed by way of example in table 1 by using an antiviral agent against the viruses. As stated above those antiviral agents which are directed against the viruses that are causally involved in the tissue change, i.e. are active, are generally suitable for this purpose. The viruses are the viruses described herein i.e. HMGI viruses and/or DNA viruses. A preferred subgroup of the viruses described herein are those viruses where the individual virus has a nucleic acid or codes for a nucleic acid which contains at least one binding site for a gene product of genes of the HMGI(Y) family or derivatives thereof or which codes for a gene product that interacts with at least one gene product of genes of the HMGI(Y) family or derivatives thereof, and belong to the group of DNA viruses.

In this connection within the group of DNA viruses, the adenoviruses appear to be of particular importance for this. A further group of DNA viruses which are of particular importance for the tissue changes described herein involving a tissue of mesenchymal origin, are viruses of the herpes group which are referred simply in the following as herpes viruses. In addition papova viruses belong to the group of DNA viruses which are of importance for the tissue changes described herein. The present invention is also based on the finding that for the tissue changes described herein various viruses and in particular DNA viruses can act synergistically.

Hence the antiviral agents according to the invention which are directed or are effective against the viruses described herein which also include vaccines directed against these viruses, prevent viral infection of the corresponding tissue or prevent multiplication of the viral material and thus prevent the formation of the complex comprising viral nucleic acid and gene products of the genes of the HMGI(Y) family and as a consequence the formation of tissue changes does not occur.

In the pathogenesis of the tissue changes described herein it also appears that gene products of the viral nucleic acid bind to gene products of genes of the HMGI(Y) family and derivatives thereof and this increases the effect of the viral transforming proteins. The various antiviral agents ultimately prevent or at least reduce this interaction.

Binding of a gene product of genes of the HMGI(Y) family or derivatives thereof to the viral nucleic acid or to the gene product of a viral nucleic acid is understood herein to include any interaction of the participating molecular species which, among others, can have the consequence that the components forming the complex can no longer be observed as individual components but rather that the complex is the only observable component which does not exclude individual components from still being present in a non-bound form. Such a binding for example includes interaction by electrostatic attractive forces, van der Wals forces, hydrophobic interaction, hydrogen bonds and disulfide bridges and combinations thereof. Such a complex comprises at least the two gene product species i.e. a gene product of the viral nucleic acid and a gene product of the genes of the HMGI(Y) family which can directly interact with one another, but can also additionally comprise a nucleic acid in which case two gene product species can interact directly but this does not necessarily have to occur.

In this respect one aspect of the invention also concerns a vaccine against transforming viruses which is suitable for the prevention and/or treatment of the tissue changes described herein.

Basically all the agents disclosed herein are suitable for the treatment as well as for the prevention of tissue changes of the type described herein. In this connection those agents are quite especially advantageous for prevention which contain a vaccine against the viruses described herein i.e. HMGI viruses and/or DNA viruses, which thus make the previous unsatisfactory treatment and prevention concepts obsolete with their not inconsiderable risk potential. In this connection it is particularly noteworthy that the use of vaccines is particularly gentle on the body since they are usually associated with comparatively fewer side effects.

The production of vaccines against viruses is generally known in the prior art and described for example in Modrow, S.; Falke, D.; “Molekulare Virologie”, Spektrum, Akad. Verl., 1997.

Basically various types of vaccines are known i.e. live vaccines which contain viruses capable of multiplication, vaccines made of inactivated virus in which the viruses are present in a form in which they are no longer capable of multiplication, cleaved vaccines which only consists of the components of the virus that are important for immunization which are also referred to as subunit vaccines or antigen vaccines and so-called “synthetic antigen vaccines”. All above-mentioned types of vaccine can be used within the scope of the present invention.

Live vaccines contain viral strains capable of multiplication which result in a specific protection but do not lead to a disease in healthy animals. A live vaccine can either be produced from homologous (of the same species) or heterologous (from a foreign species) viral strains. Viral strains that have been obtained naturally or artificially can be used as homologous vaccines.

Viral strains for vaccines that have been obtained naturally are derived from field strains which only have a weak virulence or which are no longer virulent i.e. do not cause disease in healthy animals but multiply in the host and result in the development of immunity.

A subgroup of live vaccines relates to the artificially weakened, attenuated strains. They are obtained from fully virulent field viruses that produce good immunity by artificial culture, preferably in cell cultures, which results in them losing their virulence for the natural host after a greater or lesser number of passages. The loss of virulence in such a passaged virus population does not occur simultaneously in all virus particles. However, it is possible to isolate attenuated virus particles by certain selection methods (e.g. plaque method, end dilution method etc.).

Attenuation means a specific weakening or abolition of the virulence of a replicable virus for a certain host while retaining its ability to multiply, the antigenicity and the immunogenicity remains constant over a certain succession of generations.

The attenuation can be a modification or a mutation which leads to a loss of the disease-making properties i.e. the transforming properties in the present case. It is accordingly more or less stable. Whereas in the past passages in animals or fertile eggs were carried out in the past in order to artificially attenuate the virulence, nowadays attenuated strains are mainly obtained from cell cultures by means of continuous passages.

Live vaccines from heterologous viral strains can produce immunity when there is a very close immunological relationship between different species of virus. It is then possible to use the related heterologous and thus non-disease-making viral strains as the vaccination strain.

Live vaccines have advantages and disadvantages. On average they result in an improved immunity which is of longer duration. A vaccination virus that has been well attenuated can only stimulate the immunity mechanism when it is administered in an adequate concentration. Such an adequate concentration can be determined by simple routine experiments which are within the scope of the ability of an average person skilled in the art. The vaccine protection usually already begins a few days after the vaccination. Several processes are responsible for this: interference, interferon formation, rapid development of cellular immunity. Another advantage of live vaccines is that they can be very easily applied locally e.g. orally or by aerosol. This is particularly advantageous with regard to the comparatively good accessibility of the infected tissue and organs in the case of the tissue changes described herein since in such a case the end user can apply such an agent himself.

In addition it is possible according to the invention to use vaccines comprising inactivated viruses. Virus inactivation generally refers to abolishing the infectiousness of a virus particle. In vaccine production inactivation is understood to mean that the ability of viruses to multiply is artificially removed without adversely affecting the other activities and especially the antigenic and immunogenic capability. The term “antigenic vaccine” is still sometimes used in the literature for vaccines made of inactivated viruses.

Virus suspensions of fully virulent, well-immunizing viral strains that have been manufactured, purified and highly concentrated from organs or tissues containing virus but nowadays primarily from cell cultures are used as the starting material for these vaccines. These concentrated virus suspensions are then gently inactivated by suitable processes. Chemical or physical treatments are optimal which destroy the viral nucleic acid as the carrier of the ability to multiply and destroy the infectiousness but do as little damage as possible to the protein components of the virus which are the active components for antigenicity and immunogenicity. Well-proven agents are for example formaldehyde and certain detergents. Heat and radiation are used as physical components.

In addition it is possible to use cleaved vaccines in connection with the present invention which contain antigenic and immunizing viral components which are usually in a purified form and are obtained by cleaving viruses.

In the case of the coated viruses these are the virus-specific glycoproteins of the lipid-containing coats. Specific antibodies to these antigens bind in vivo to the glycoproteids of the virus, thus blocking their ability to adhere to cells and hence their infectiousness.

The immunizing glycoproteids of coated viruses can be released by chemically cleaving the virus coat (particular lipid-dissolving detergents). As a result the virus spontaneously and completely loses its infectious properties and subsequently the desired glycoproteid is purified of undesired components such as nucleoprotein, capside enzyme, lipids etc. in the cleaved viral material by physico-chemical methods and is then concentrated and processed to form the vaccine.

In the case of uncoated naked viruses it is possible for skilled persons to develop cleaved vaccines from the capsid proteins that are of major relevance for the immunization.

Finally it is also possible to use synthetic antigenic vaccines which have been produced by genetic engineering. By isolating and identifying the relevant genes of the viral genome it is for example possible to produce the capsid protein of the corresponding viruses by genetic engineering. In this connection a person skilled in the art would have the ability to truncate the appropriate nucleic acid and the proteins resulting therefrom to such an extent that only the antigenic determinants or the appropriate epitope remains as the vaccine component. The device according to the invention can also be used in this sense. The viral nucleic acid bound to the carrier material carrying the gene product of genes of the HMGI(Y) family can, usually after elution, be used to determine which virus or nucleic acid thereof is involved in the formation of the complex of viral nucleic acid and gene product of a gene of the HMGI(Y) family that is responsible for the formation of the tissue changes described herein.

After this identification the device according to the invention can, however, also be used to provide an essential component for the production of a preparation according to the invention for preventing and/or treating the tissue changes described herein. For this purpose a viral nucleic acid or a pool of viral nucleic acids is added to the device according to the invention, whereupon the viral nucleic acid having at least one binding site for the gene product of a gene of the HMGI(Y) family binds to the device or to the gene product of a gene of the HMGI(Y) family that is bound to a carrier material in this device. Non-bound or unspecifically bound. viral nucleic acid is removed for example by washing the carrier material with a suitable wash solution. Subsequently the specifically bound viral nucleic acid is eluted from the carrier material. The nucleic acid obtained in this manner can then be used to form a viral component which is used in the preparations according to the invention. For example the viral nucleic acid can be cloned into an expression vector and the expressed viral peptide or protein can be used as the vaccine. Alternatively the viral peptide or protein expressed in this manner can for example, after further optional intermediate steps which are known to a person skilled in the art, be used to produce antibodies which can then in turn be used as a vaccine in the preparations according to the invention. In this connection the expressed viral peptide or protein or the antibodies directed against them are essential components for the production of a preparation to prevent and/or treat the tissue changes described herein.

The same also applies to the case in which a gene product of the viral nucleic acid is bound to a gene product of a gene of the HMGI(Y) family. In this case, instead of the viral nucleic acid, the gene products of the viral nucleic acid, in particular of a nucleic acid of candidate viruses i.e. those viruses which are presumed to be causally involved in the formation of the tissue changes described herein, are added. The gene products coded by the viral nucleic acid that are obtained after specific binding and subsequent elution can then be used directly as a vaccine or be subjected to further modification or further processing steps. This may also include further purification of the vaccines or adding appropriate adjuvants or treating or preparing the vaccine in a suitable manner for the intended use.

Adjuvants such as complete or incomplete Freund's adjuvant can be added to the vaccines. Other additives are known to a person skilled in this field.

In general the preparations according to the invention can be present as a pharmaceutical preparation which, in addition to at least one of the various preparations according to the invention, also contains a suitable pharmaceutically acceptable carrier. Suitable pharmaceutically acceptable carriers are known to a person skilled in the art.

The preparations according to the invention can contain other additives which for example stabilize the preparation, act as preservatives or modify the properties of the preparation and substances which modify the properties of the preparations according to the invention.

Such substances which modify the properties of the preparation according to the invention itself can for example in the case of vaccines be adjuvants such as incomplete or complete Freund's adjuvant.

The preparations according to the invention can have other components which convert them into a preferred form for the respective intended form of administration. Thus for example if the preparations according to the invention are in the form of aerosols, a vehicle necessary for aerosol formation can be provided in addition to the actual preparation.

The preparation according to the invention can for example be present in the form of a solution or emulsion for intradermal, intravenous or subcutaneous injection. Liquid solutions of the preparations according to the invention can also be in an infusible form.

Furthermore the preparations according to the invention can also be present in a lyophilized form.

Furthermore the preparations according to the invention can in principle be present in any pharmaceutically suitable form including for example tablets, dragees, suppositories, gels, powder.

If the preparation contains a vaccine, the vaccine itself can be a complete virus particle which is alive or attenuated, or inactivated or contains parts thereof, the parts thereof typically carrying the antigenic and immunological properties of the respective virus. The vaccine may contain numerous different virus particles or antigenic or immunogenic parts thereof. The virus particles and the parts thereof can themselves be present in a modified form. Such modifications can be in the forms that are generally known for peptides, proteins which are optionally glycosylated.

The virus particles or parts thereof can be produced by genetic engineering. The virus particles or parts thereof do not have to be identical with the viruses that are responsible for or at least associated with the formation of the tissue changes described herein. It is important that the viruses, including parts thereof, that are used in the vaccine or its production are suitable for generating an immune response directed against the causal agent for the disease i.e. virus and thus block the transforming properties of the agent or virus involved in the pathogenesis.

The above-mentioned also applies in the same sense to the case that the vaccine contains an antibody.

It has been found that the binding site for a gene product of genes of the HMGI(Y) family or derivatives thereof on the nucleic acid of the virus consists of a number of structural and sequence features. The presence of a first AT-rich sequence appears to be of fundamental importance for the binding of gene products of the HMGI(Y) family or parts thereof and derivatives thereof. Such an AT-rich sequence is not to be understood as only a sequence of AT dimers. On the contrary, the two bases can be in any order in this sequence and can be interrupted by individual nucleotides or several nucleotides. In addition to this first structural and sequence feature, the said binding sites for the gene product of the HMGI(Y) family additionally have a second AT-rich sequence which can have a similar structure to the first AT-rich sequence, Both AT-rich sequences are arranged at a spatial distance relative to one another. This spatial distance results from the spatial dimensions and thus from the secondary and tertiary structure of the HMGI(Y) proteins i.e. the gene products of the genes of the HMGI(Y) family. As a consequence of the secondary and tertiary structure it is necessary that the first sequence and the second sequence are arranged relative to one another in one plane on the viral nucleic acid for the HMGI(Y) gene product to bind. If one regards the DNA as a three-dimensional model, the AT-rich sequences that act as binding sites are each on the side facing the observer in the above-mentioned arrangement. This arrangement is also referred to as “same face”. If these three structural and sequence features of the viral nucleic acid are present, the gene product binds very persistently to the viral sequence, especially in the regulatory regions of the nucleic acid (e.g. promoter and enhancer) since these preferably have the said structural and sequence features. If one or several of these sequence or structural features is absent, a gene product of the genes of the HMGI(Y) family will only bind weakly or not at all.

If, in contrast, one regards the secondary and tertiary structure of the gene products of the genes of the HMGI(Y) family, they have three regions or domains which are arranged at a defined distance to one another and have a high affinity to AT-rich sequences. Hence a prerequisite for a successful binding to a nucleic acid is that the AT-rich sequences are arranged at a corresponding distance. This distance results from the pitch of the nucleic acid which is usually ca. ten base pairs which means that the spacing of the AT-rich sequence is usually 10 base pairs or integral multiples thereof of up to ≦3.

In addition to vaccines in general and especially those vaccines against the viruses described herein i.e. HMGI viruses and/or DNA viruses, according to the invention those vaccines are suitable for the agents according to the invention which are directed against an above-mentioned virus the nucleic acid of which contains at least one binding site for at least one gene product of genes of the HMGI(Y) family or derivatives thereof. The genes of the HMGI(Y) family are well-known in the prior art.

The genes of the HMGI(Y) family represent a family of genes which comprise among others HMGIC, HMGIY and MAG genes. Reference is made by way of example to the international patent applications PCT/EP96/00716 and PCT/DE96/02494 the disclosed contents of which are incorporated by reference. It is remarkable that the binding site on the viral nucleic acid is suitable for any of the previously mentioned genes or gene products thereof. The term gene product as used herein is also intended to refer to parts thereof provided these parts are still suitable for binding a viral nucleic acid. Similarly the term gene product is intended to include the respective derivatives of the gene product which have a modification like that which can be commonly carried out on peptides and proteins and for example comprises deletions and substitutions of the carboxy-terminal sections of the proteins. In particular the term derivative of the genes of the HMGI(Y) family also includes the aberrant transcripts and the translation products thereof described in PCT/DE96/02494, provide that these are still able to bind to the viral nucleic acid especially in regulatory regions of the nucleic acid (e.g. promoters and enhancer). The characterization of such aberrant transcripts is described for example by Kazmierczak, B. et al; Am. J. Pathol., 1998; 153(2): 431-5 and their structure is for example described by Schoenmakers, E. F. et al, supra.

In an alternative embodiment of the preparation according to the invention the vaccine can be an antibody which is directed against the virus or at least against one of the viruses described herein. In this connection it is sufficient when the antibody is not primarily directed against the virus or a corresponding part thereof but fulfils its function by mediating an appropriate cross-reactivity and thus ensures that no interaction occurs between the viral nucleic acid and the gene product of the genes of the HMGI(Y) family in the sense that the pathogenicity mechanism described above and the described tissue changes occur. In this connection the antibody can be directed against any desired component of the virus i.e. it can be directed against or interact with proteins or protein fragments of the virus capsid, the glycopeptide which may be present or the corresponding viral nucleic acid or fragments thereof.

The antibody used within the scope of the invention can be a monoclonal antibody or polyclonal antibody. The term antibody as used herein can encompass a mixture of different monoclonal antibodies. In addition the term antibody is intended to include any peptide or protein which has at least one antibody property and in particular binds to a suitable epitope and ensures that the complex of antibody plus epitope or antigen is converted into a form which can no longer participate in the pathogenesis of the tissue changes described herein or is removed from the pathogenesis process. This can for example be achieved by ultimately preventing an interaction of a gene product of a gene of the HMGI(Y) family with the viral nucleic acid or with a gene product of a viral nucleic acid (in which case the corresponding virus is causally involved in the formation of the tissue changes described herein); this prevention can be direct or indirect whereby an example of indirect prevention is to remove the corresponding viruses or virus particles. The term antibody also includes antibody fragments and derivatives thereof and in particular (Fab)′ or F(ab)₂ fragments and single-chain antibodies and such derivatives. Derivatives of these antibodies that are also known to a person skilled in the art.

The above-mentioned also applies in the same sense in the case that a vaccine is provided against a virus whose nucleic acid codes for a gene product wherein this gene product interacts with at least one gene product of genes of the HMGI(Y) family or derivatives thereof.

The viruses against which the vaccine or antibody in the preparation according to the invention is directed belong to various groups and types of viruses described herein.

When the preparation according to the invention is used to immunize and in particular to actively immunize against the viruses which are associated with the pathogenesis and/or aetiology of the tissue changes described herein, the viruses are preferably those which are causally connected or associated with the pathogenesis or aetiology of the tissue changes described herein.

Cell cultures are used in the methods according to the invention which are derived from a tissue change or parts of the tissue or tissues thereof. The preparation of such cell cultures is known to a person skilled in the art and is described for example in Stem C. et al., “Geburtsh. u. Frauenheilk. 52 (1992), 767-772.

A normal karyotype is understood herein as the set of chromosomes that is obtained by using routine techniques provided it exhibits no detectable anomalies especially in the regions 12q14-15 and 6p21 in an image of ≧500 bands per haploid set.

An expression vector for a gene of the HMGI(Y) family is characterized by the fact that in its totality it leads to the expression of a gene of the HMGI(Y) family and thus to the production of corresponding gene products. Such an expression vector typically contains an origin of replication, the nucleic acid coding for a gene product of the HMGI(Y) family or a fragment thereof and suitable transcription and translation regulation sequences. Such constructs are known in the prior art and are described for example in Winnacker, E.-L.; From Genes to Clones; Weinheim; New York: VCH, 1987.

In principle any transfection method which leads to a transfection of the appropriate cell cultures can be used for the present invention.

The preparation of cDNA is known to persons skilled in the art. In order to compare the RNA or cDNA pattern of the transfected cells or cultures with those of non-transfected cells or cultures one typically carries out a so-called differential display method (Diatchenko, L.; Proc. Natl. Acad. Sci. USA, vol. 93, p. 6025-6030, 1996).

The procedure for examining by sequence homology whether viral elements are present in the RNA(s) that are expressed or more strongly expressed in the transfected cultures compared to control cultures is to check and optionally identify the sequence with the aid of relevant data banks e.g. BLAST, a data bank service of the National Center for Biotechnology Information (NCBI).

In the scope of the method according to the invention it is also possible to use the nucleic acids derived from the primary transcription products to compare the RNA pattern of the transfected cultures with those of control cultures. It is correspondingly possible to make this comparison on the basis of the cDNA pattern in which case the cDNA of the transcription products, i.e. of the RNA species, is produced by using known methods.

The control cultures usually have a normal karyotype and are derived from a tissue or part of a tissue which is contained in the changed tissue described herein and which are transfected with an expression vector that, however, lacks the gene for the HMGI(Y) family or a derivative thereof i.e. lacks the insert coding for a gene product. Control cultures can also be those which are not transfected with any expression vector at all.

In a further method according to the invention in which a PCR test is carried out and a viral probe is used for the PCR, the PCR test is carried out according to known methods in the prior art. A PCR test is understood as a polymerase chain reaction test in which at least one sequence-specific primer is used to selectively amplify the required nucleic acid or the required nucleic acid fragment (see Newton, C. R.; Graham, A.; “PCR”; 1994, Spektrum Akadem. Publishers, Heidelberg, Berlin, Oxford).

Primers or viral probes are understood herein as oligonucleotides and/or nucleic acid fragments which can be used to detect nucleic acids which have a homology to the primer or probe. Such viral probes can be prepared by any methods known to a person skilled in the art such as by organic-chemical synthesis or by using PCR or cloning techniques.

If a cDNA library of a tissue change as described herein or a part thereof is set up within the scope of the method according to the invention, in which a gene of the HMGI(Y) family or a derivative thereof is activated, the activation can be recognized by a corresponding chromosomal aberration.

In the method according to the invention the screening is typically carried out under conditions of low stringency. Conditions of low stringency are understood in this connection to mean that by reducing for example the hybridization temperature or modifying the wash conditions (e.g. increasing the salt concentration) binding also occurs to those nucleic acids which have considerably less homology to the sequence of the probe whereby at first the co-detection of false-positive nucleic acid sequences is accepted since the final clarification of whether it is a positive signal or result is achieved or can be achieved by sequencing and sequence analysis of the identified sequences and thus allows the false-positive sequences to be eliminated.

After it has been found that viral elements are present in the tissue that has changed as described herein or in the cell cultures derived therefrom by comparing the RNA or cDNA pattern of transfected cell cultures with that of control cultures, or by a positive signal in a PCR test using primer (pairs) which correspond to sequences of viral nucleic acids, or by a positive signal when screening a cDNA library with a virus-specific probe, or by analysing the cDNA clones for viral sequences or comparison with a cDNA library from a normal mesenchymal or optionally epithelial tissue, in the method according to the invention can also provide for the viral elements to be identified and/or classified and used solely for the vaccine production.

The device according to the invention for determining a virus involved in the pathogenesis comprises a gene product of genes of the HMGI(Y) family which is bound to a carrier. In connection with the device according to the invention and its possible uses the term gene product also includes parts of the gene products or derivatives of the gene products. In this connection the definition of gene products is also based on the definition given above for genes of the HMGI(Y) family.

The gene product is coupled to a carrier material which can be appropriately selected by a person skilled in this field. Any materials are suitable as carrier materials which allow a binding of proteins or derivatives whether this is by means of a direct or indirect binding. Suitable carrier materials can be typically found among chromatographic materials. Such a binding can also be an adsorption or a reversible binding. Since the gene products of the HMGI(Y) family are proteins, the methods and compounds known to a person skilled in the field for immobilizing proteins can be used.

With regard to how the gene product or gene products are bound to the carrier material, special care must be taken that the region responsible for binding to the viral nucleic acid is bound to the carrier material or is available for binding viral nucleic acid. Hence the corresponding gene product can be truncated or for example be provided as a fusion protein. In this connection any construct is conceivable provided the part of the gene product or the part of the gene product of the genes of the HMGI(Y) family that binds nucleic acids and is responsible for binding the gene product of the viral nucleic acid, is still available for binding a nucleic acid.

In a concrete case such a device can be designed such that it is an affinity chromatographic column and at least one gene product of genes of the HMGI(Y) family which can be one gene product or different gene products, is immobilized on the column material and various preparations or mixtures of viral nucleic acid or of a gene product coded by the viral nucleic acid is applied to the carrier matrix. As a result of the interaction between the gene product and the viral nucleic acid or the gene product of the viral nucleic acid, a stable complex is formed. Non-specifically bound viral nucleic acid or unbound nucleic acid or non-specifically bound or unbound gene product of viral nucleic acid and other components of the applied sample are washed from the column. Afterwards the interactions between the gene product and the viral nucleic acid bound to the gene product or the bound gene product of the viral nucleic acid are reduced, optionally specifically, by a suitable elution buffer such that the appropriate viral nucleic acids or the gene products coded by these nucleic acids are eluted and can be further analysed.

The invention is further elucidated by the following examples, experimental results and figures.

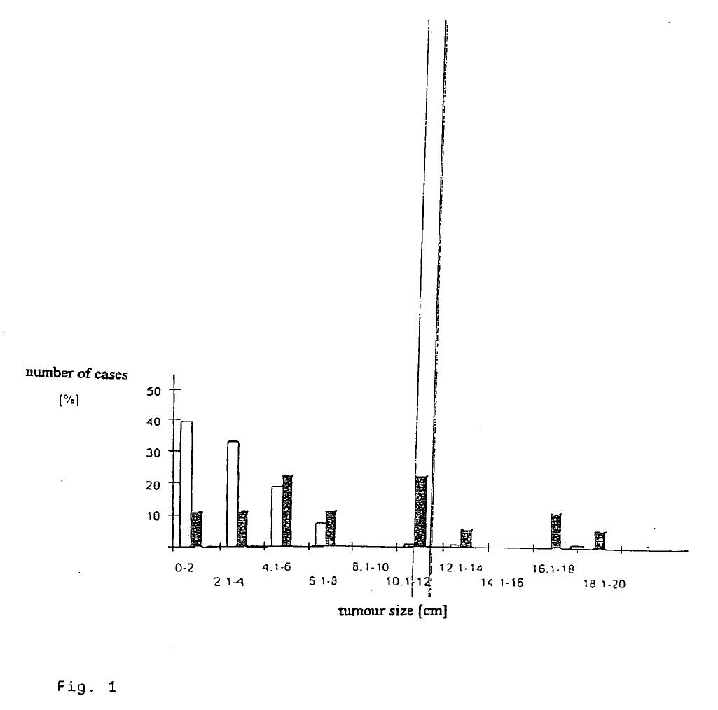

FIG. 1 shows the size distribution of myoma of normal karyotype (grey columns) and having 12q 14-15 aberrations (black columns);

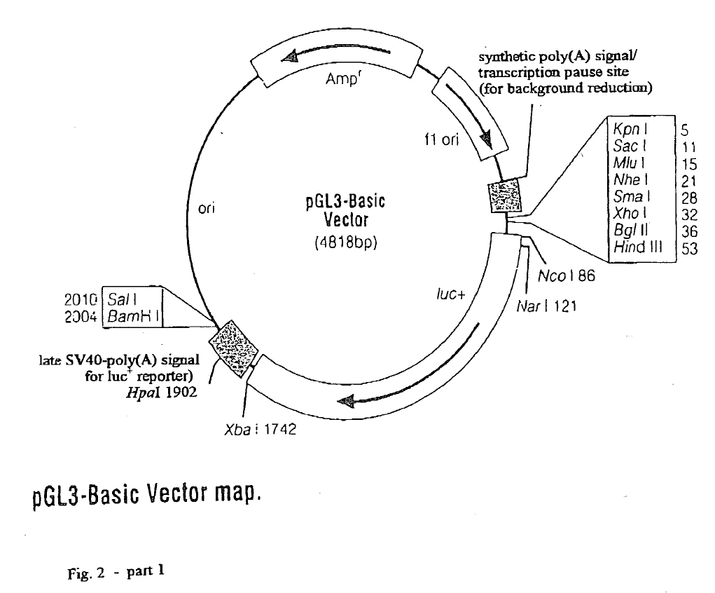

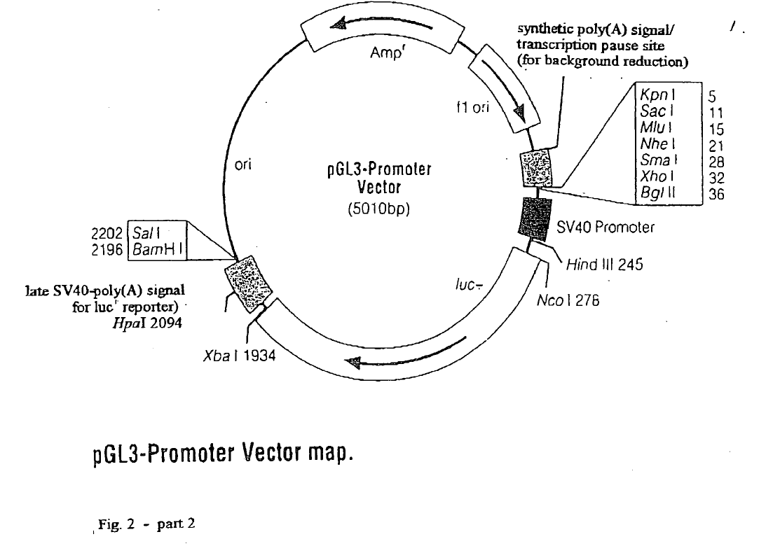

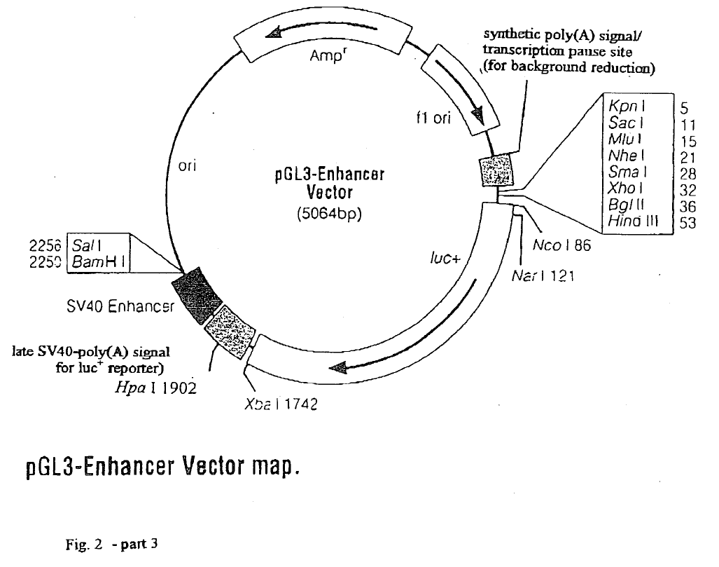

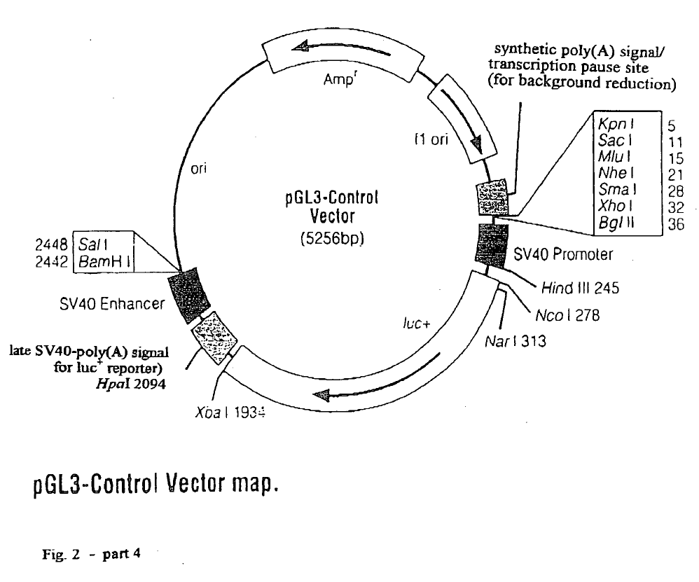

FIG. 2 shows a list of vectors used in example 4;

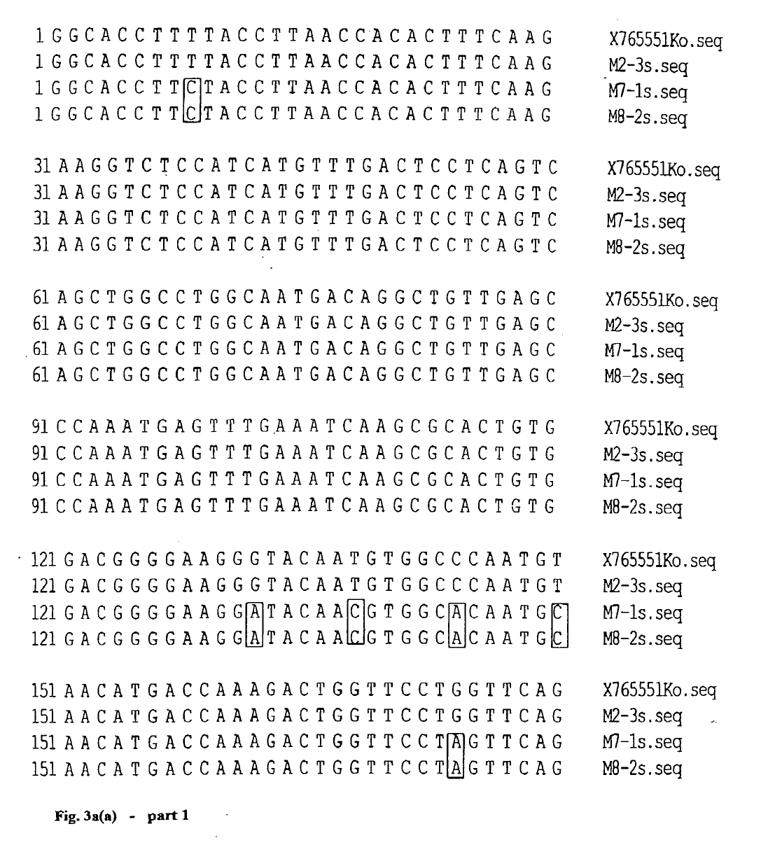

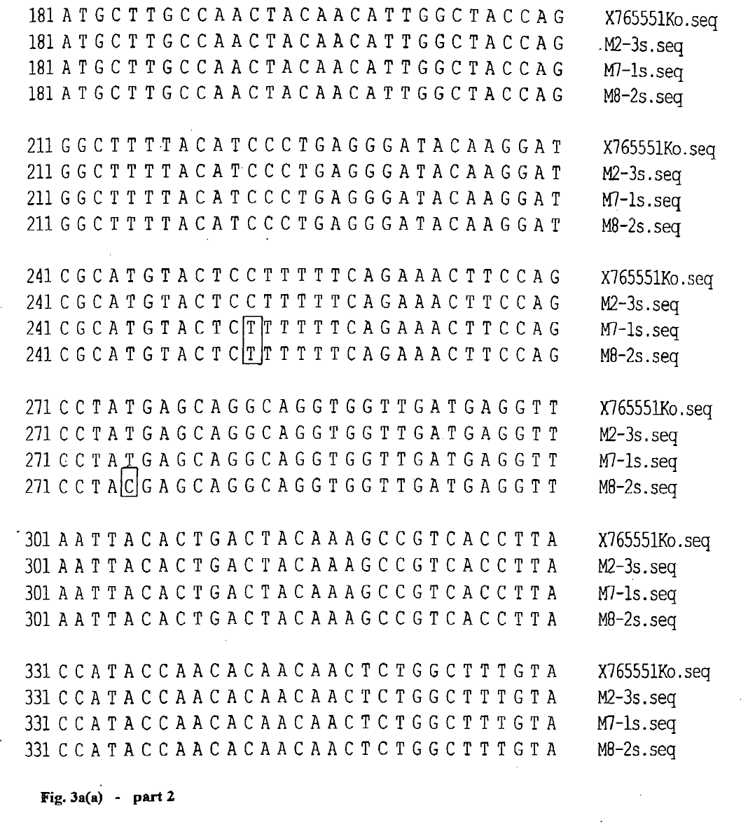

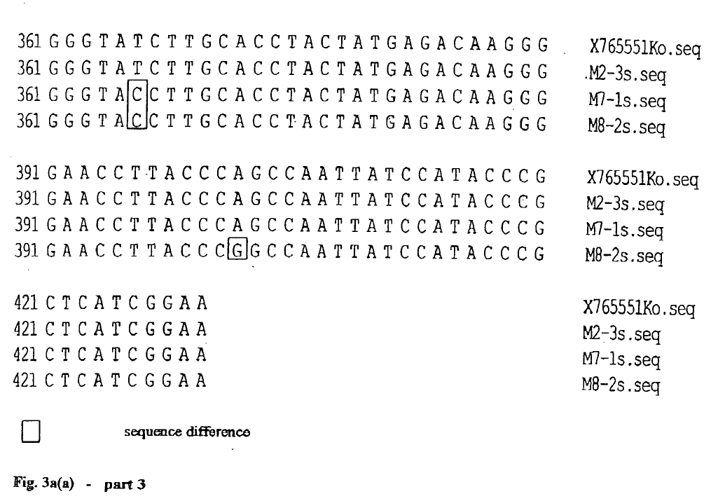

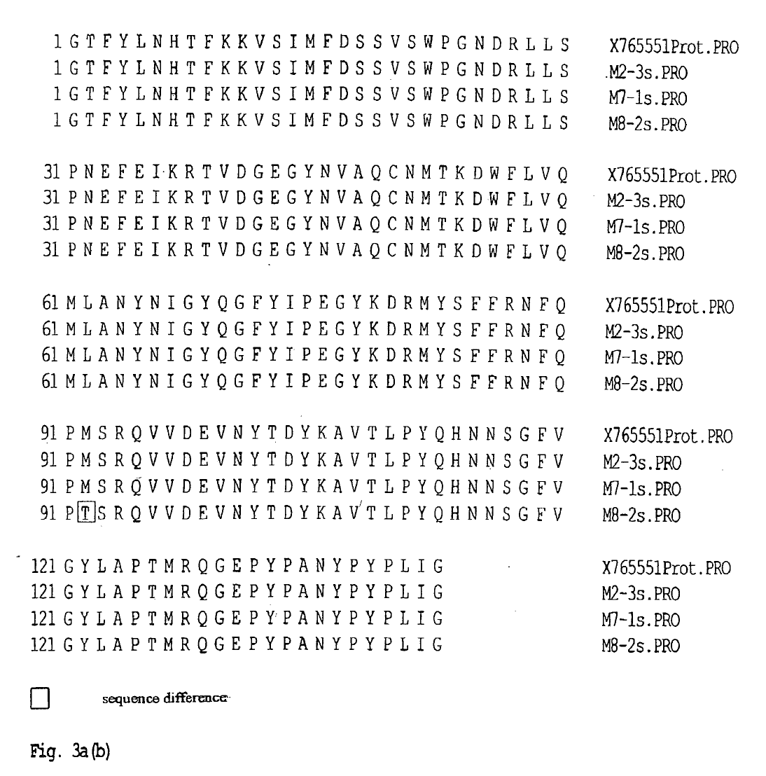

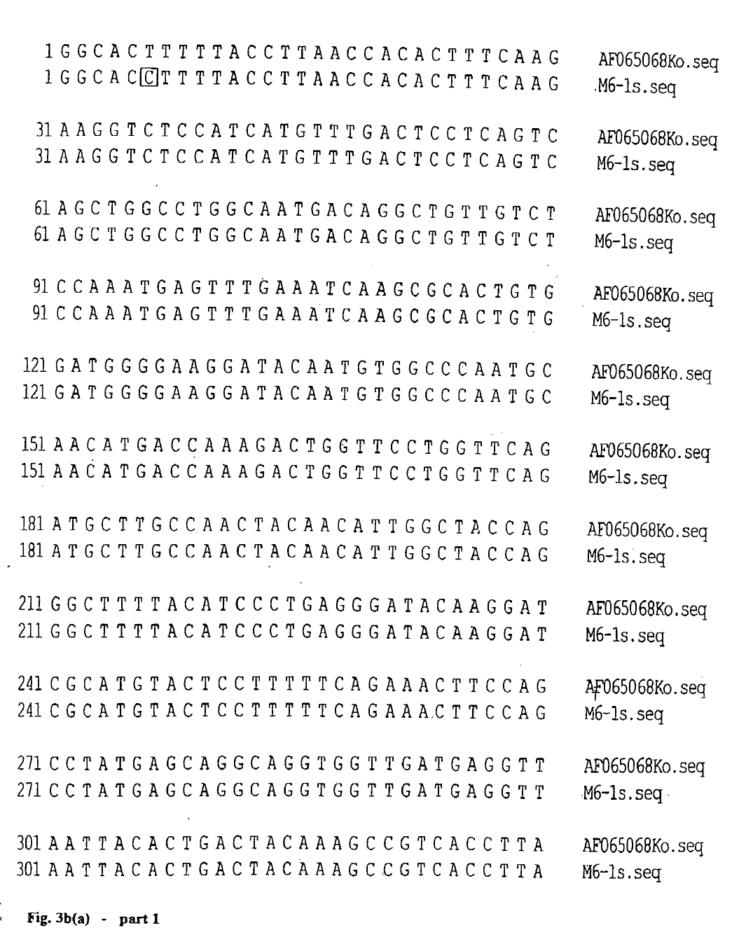

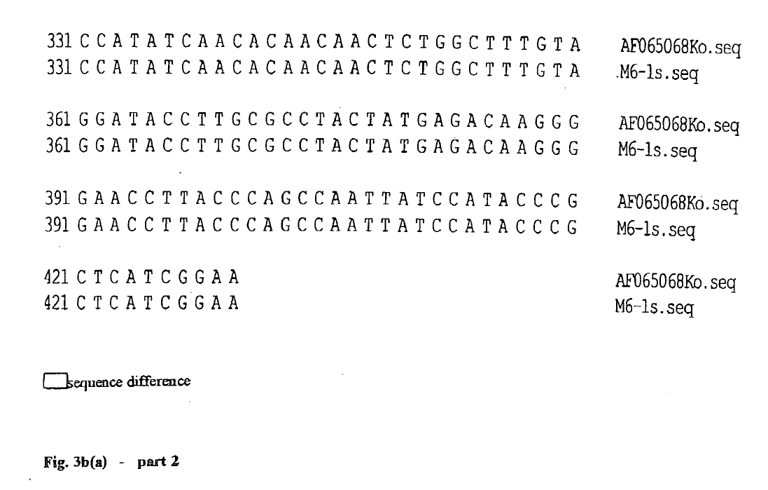

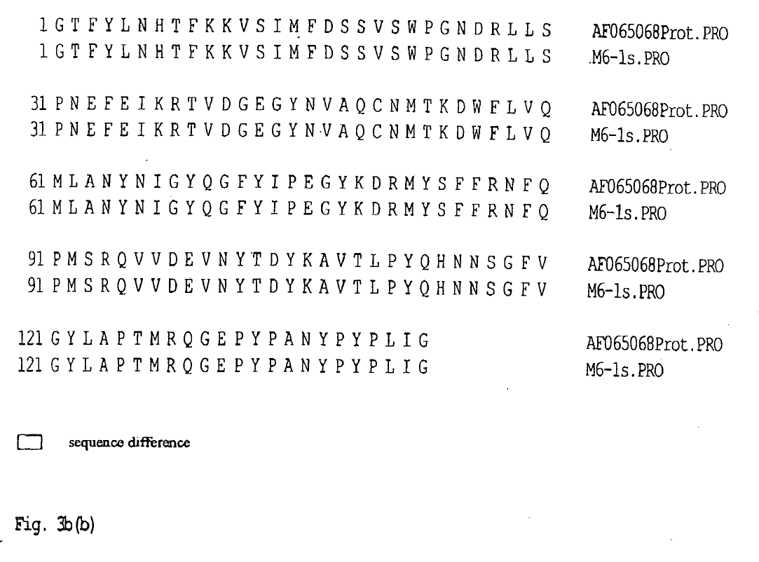

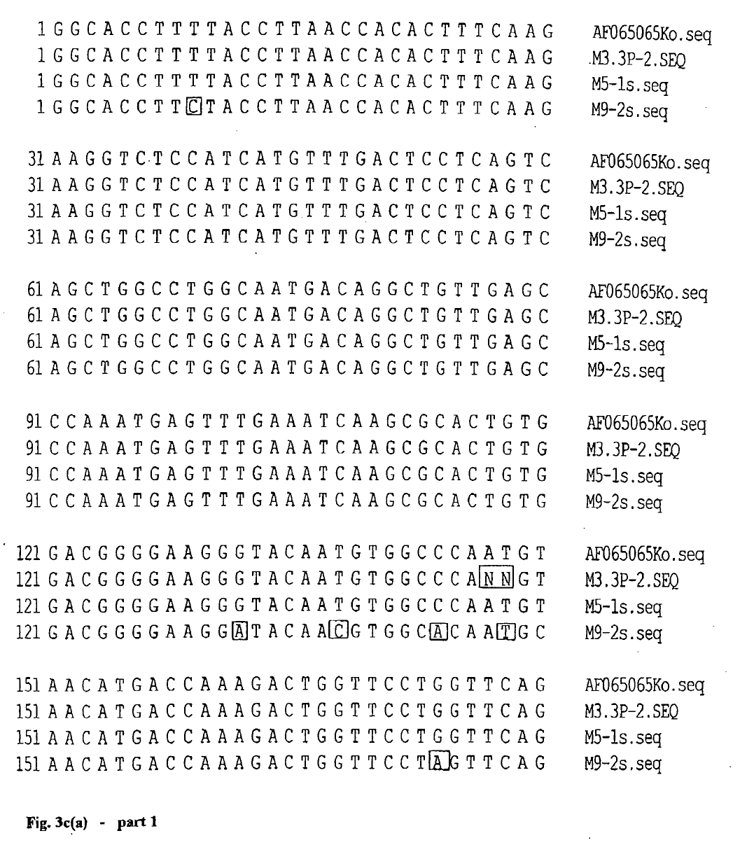

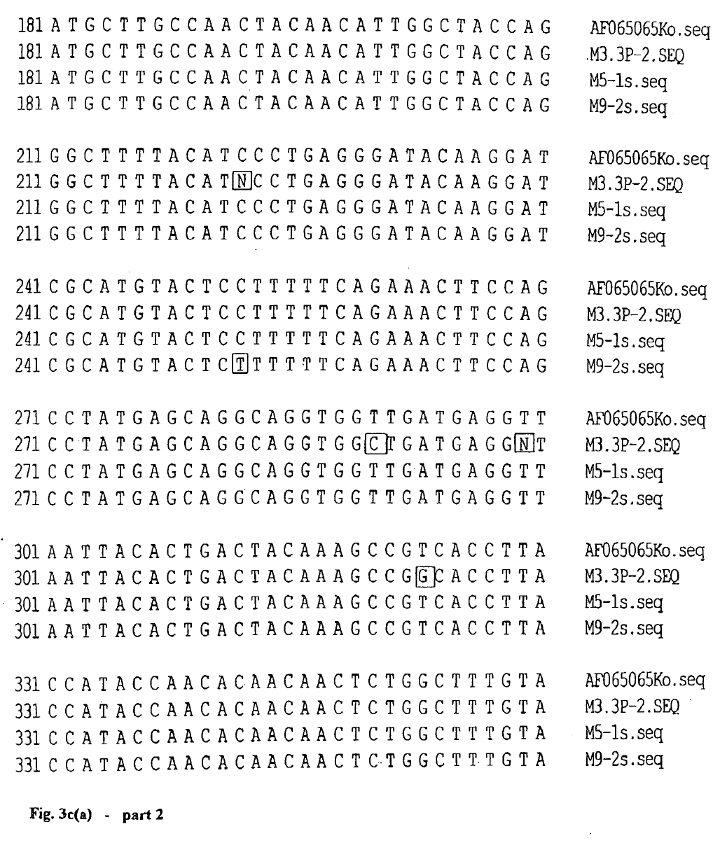

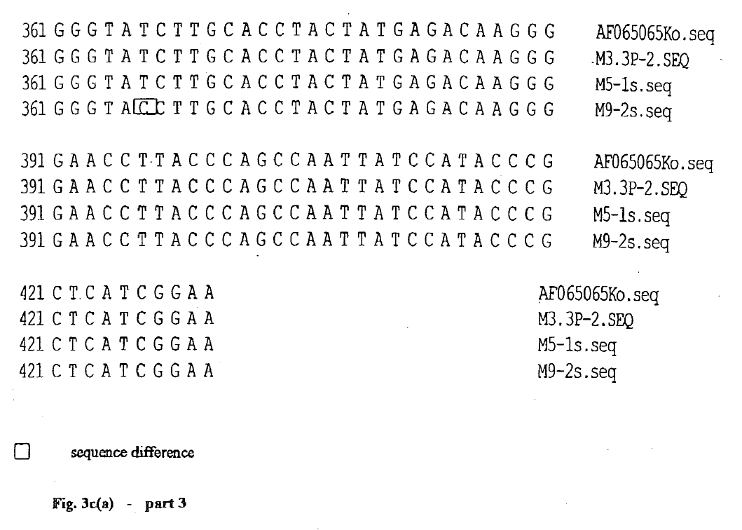

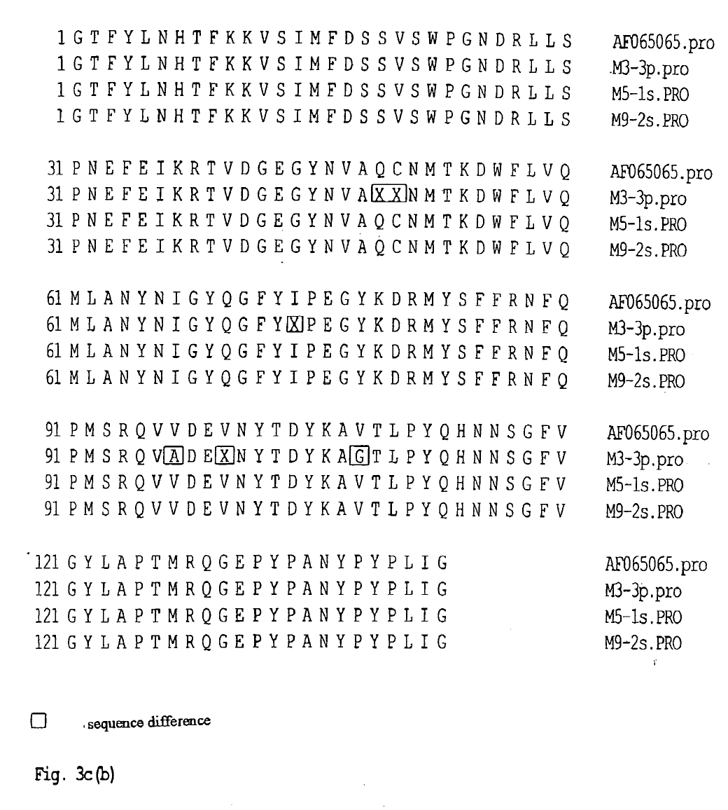

FIG. 3a-c show a sequence comparison of various sequences of myoma tissue with adenoviral sequences;

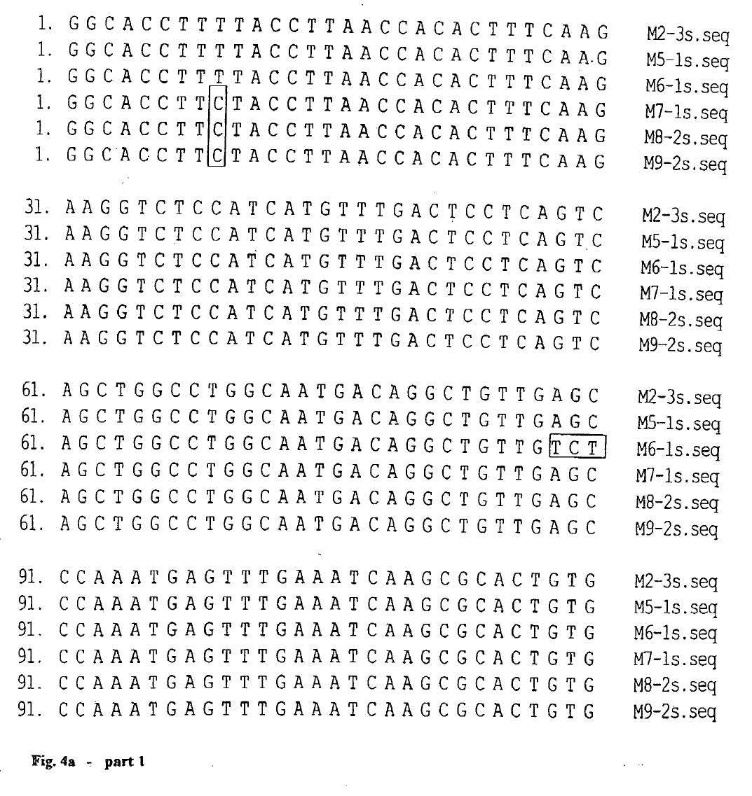

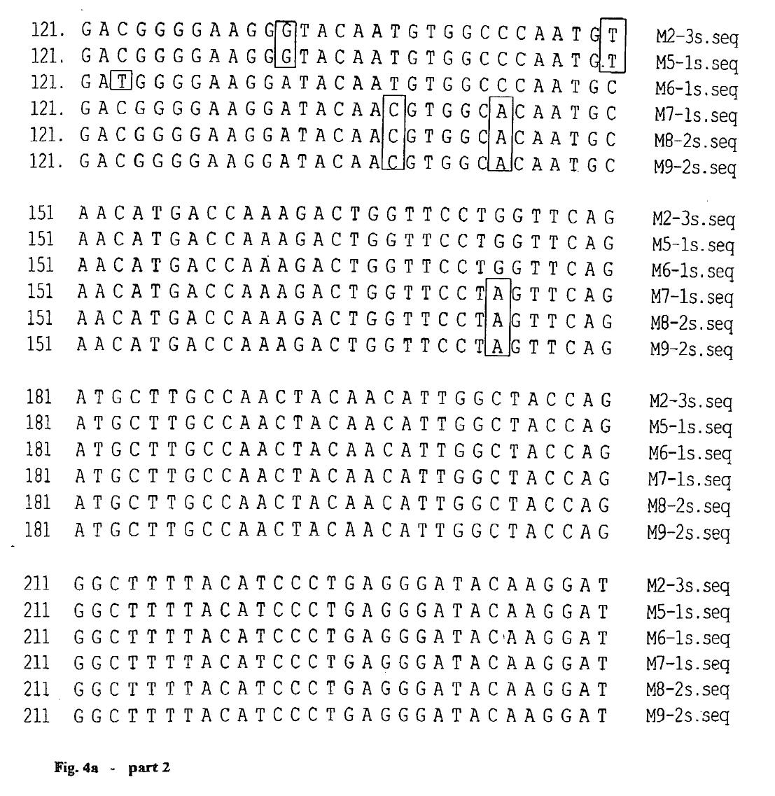

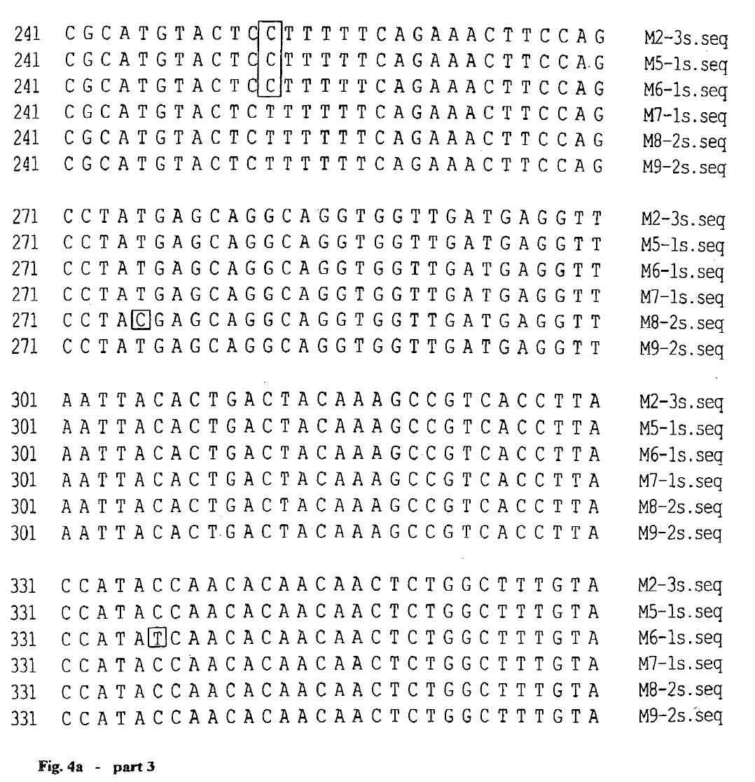

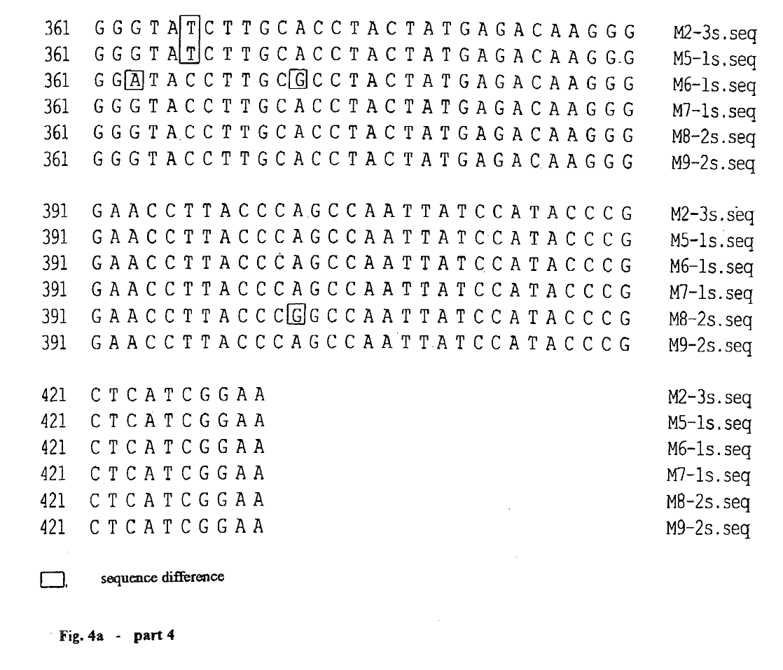

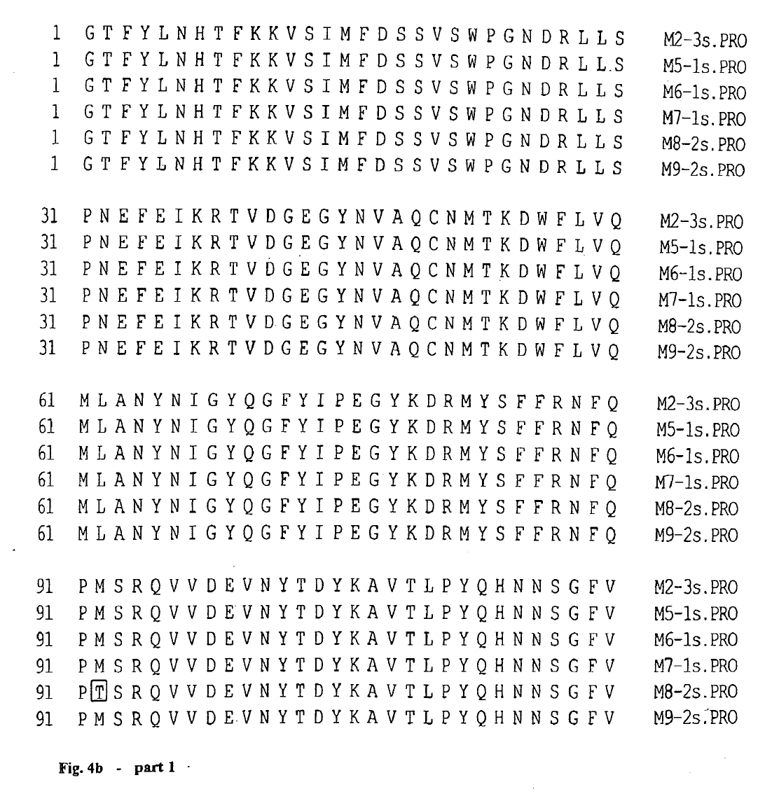

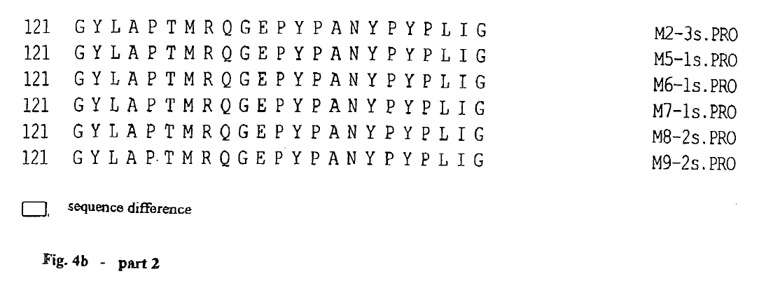

FIG. 4a-b show comparable analyses of various sequences containing myoma tissue;

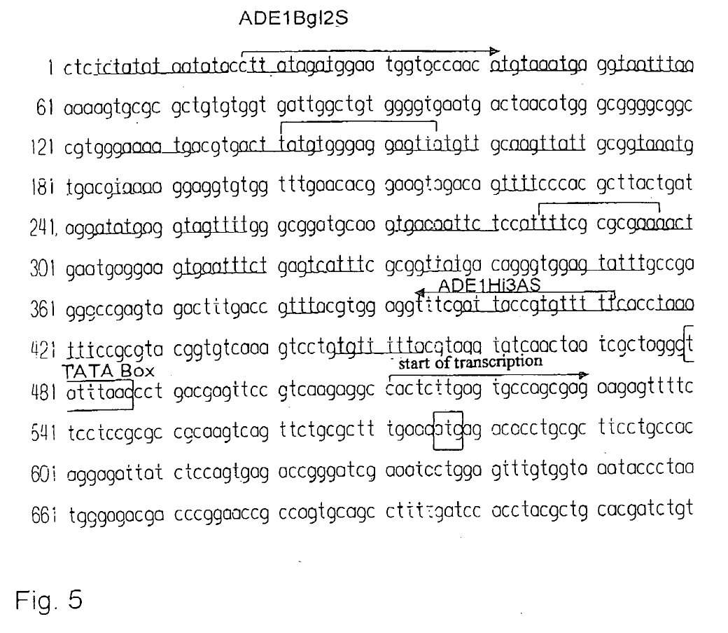

FIG. 5 shows possible HMGI(Y) binding sites in the promoter sequence of the adenoviral protein E1A; and

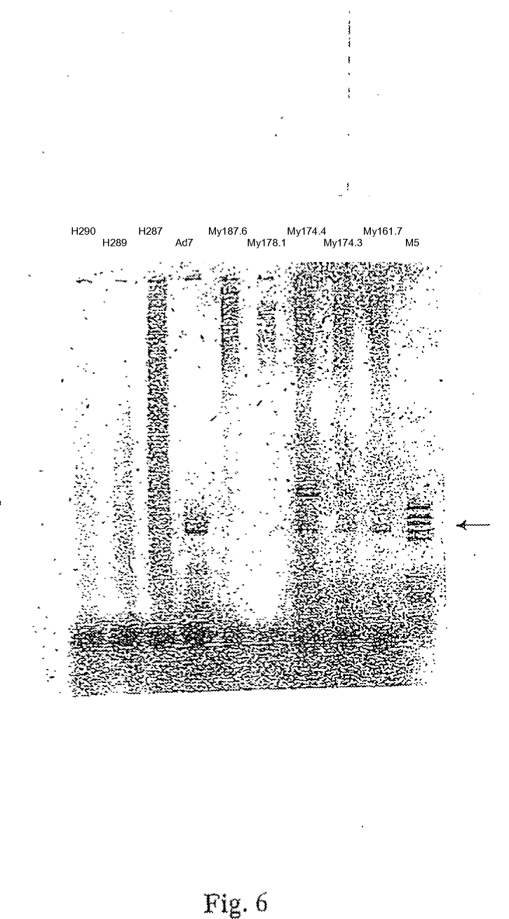

FIG. 6 shows the result of a PCR analysis which was used to examine the presence of an adenoviral DNA fragment in various tissue changes.

### EXAMPLE 1

The results of previous cytogenetic studies of uterine myomas are contradictory with regard to possible correlations between tumour size and the occurrence of clonal chromosomal aberrations. However, the problem of these studies is that the examined tumours had probably been selected according to size. Since a possible correlation also depends on whether chromosomal aberrations appear as a secondary occurrence and increase the growth potential of the tumours in question, a study was carried out using unselected myomas. In this case myomas were only examined after hysterectomies and all tumours were examined that had been detectable by palpation of the surgically removed uterus.

A total of 155 myomas from 96 female patients were examined cytogenetically. 28% of these myomas showed clonal changes in the karyotype. In the three main karyotype groups the relative proportion of normal karyotype, aberrations of the chromosomal region 12q 14-15 and deletion of the long arm of chromosome 7 was 72%, 12% and 8% respectively. The average tumour size of the groups is shown in table 3.

TABLE 3
Average myoma size in the three main karyotype groups
with 12q14-15 aberrations, deletion of the long
arm of chromosome 7 and normal karyotype.

| | average size [cm] ± |
|---|---|
| karyotype group | standard deviation [cm] |
| normal karyotype | 3.4 ± 2.1 |
| 12q14-15 aberrations | 8.9 ± 5.6 |
| deletion of chromosome 7 | 3.5 ± 2.0 |

The results clearly show that the occurrence of 12q14-15 aberrations which correlate molecular genetically with mutations in or in the area of the HMGIC gene are associated with a highly significant increase in the size of the corresponding myomas if one compares them either with myomas having a normal karyotype or having deletions in the long arm of chromosome 7. The differences are not only evident when comparing the means but also the distribution of tumour sizes of the individual tumour groups as shown in FIG. 1.

In summary the occurrence of chromosomal aberrations of the chromosome region 12q14-15 which is associated with increased expression of the HMGIC gene or the expression of changed transcripts of this gene apparently leads to an increase of tumour growth.

### EXAMPLE 2

Mutations in the area of the HMGIC or HMGIY gene are manifested cytogenetically by chromosomal aberrations of region 12q14-15 or 6p21. At least theoretically it is indeed conceivable that the aberrations that can be detected by cytogenetics are only the tip of the iceberg and a substantially larger proportion of the mutations of the two said genes are associated with chromosomal modifications that are not revealed by cytogenetics. If this were the case, it would support the key role of the said aberrations in the overall tumour development in the sense of a primary mutation. One method of detecting the hidden rearrangements is fluorescence in situ hybridization (FISH). FISH experiments were carried out on a series of 40 myomas having an apparently normal karyotype using cosmid and PAC probes which cover the locations of the HMGIC and HMGIY gene. The probes were selected in such a manner that a region of ca. 150 kb 5′ to 40 kb 3′ of the HMGIC gene and from 30 kb 5′ to 40 kb 3′ of the HMGIY gene were covered. All myomas were examined with the probes for both genes; in each case at least 20 metaphases were analysed. In no case was there any indication for concealed chromosomal rearrangements of the examined regions which could not be detected by conventional cytogenetic means.

If one takes into consideration the frequency of the myomas without detectable cytogenetic changes (see example 1) and the results of the investigations shown in this example, it is probable that the mutations of the gene of the HMGI(Y) family do not play a key role in the majority of uterine myomas.

### EXAMPLE 3

Irrespective of whether these changes are primary or secondary, the molecular genetic basis for the 12q14-15 and 6p21 aberrrations in the case of uterine myomas is assumed to be due to the fact that the chromosomal rearrangements lead to an expression/increased expression or expression of aberrant transcripts of the HMGIC gene or HGMIY gene which is absent in normal uterine tissue. In the case of the HMGIC gene there is usually no detectable HMGIC gene expression by means of RT-PCR in normal uterine tissue. This method was used to investigate 40 myoma tissues which apparently had a normal karyotype. The aim of the investigation was again to determine whether in these myomas like, those with 12q14-15 changes, there is any evidence for HMGIC gene expression.

In none of these myomas was evidence found for an expression so that also from these results it can be concluded that HMGIC gene expression is not the primary event in the formation of these myomas.

### EXAMPLE 4

Two vector systems are transfected into a cell in order to examine the effect of HMGIC on the SV40 promoter. These are the expression vector H₃Hₓ for HMGIC and the pGL3 luciferase reporter vector from the Promega Company. The complete pGL3 luciferase reporter vector system from Promega contains 4 different vectors which enable DNA sections to be examined for promoter or enhancer regions. These vectors are shown in FIG. 2. The vector “pGL3 enhancer” is required for the examination of promoter sections. The vector “pGL3 promoter” is used to examine enhancer elements. Furthermore the vector “pGL 3 promoter” is used in this experiment to test the mode of action of HMGIC on a SV40 promoter or promoters of other polyoma viruses. For this purpose the vector H₃Hₓ was cotransfected with the vector “pGL3 promoter”.

The vector “pGL3 control” serves as a positive control for the system and a transfection with only the vector “pGL3 promoter” serves as a negative control.

Apart from the promoter and enhancer elements, the individual vectors have the same basic structure. They have a modified coding region for firefly luciferase (Photinus pyralis) (luc+) which was selected to examine the transcription activity in transfected eukaryotic cells. In addition they contain a prokaryotic origin of replication for replication in E. coli, an ampicillin resistance gene for the selection, an origin of replication for filamentous phages (fl ori) for the production of single-stranded DNA (ssDNA) and a multi cloning site (MCS) 3′ and 5′ of the luciferase gene.

The “pGL3 promoter” vector is 5010 base pairs in size and in contrast to the pGL3 enhancer, contains an SV40 promoter and no enhancer. DNA fragments which contain putative enhancer sequences can be inserted on the 3′ or 5′ side of the luciferase gene and thus lead to an amplification. Furthermore the SV40 promoter can be replaced by other polyoma virus promoters.

The vector “pGL3 control” (5256 base pairs) contains an SV40 promoter and an enhancer sequence which in most mammalian cells results in an increased expression of luc+. This vector is used to control the transfection efficiency and is the internal standard for the promoter and enhancer activity of other vectors.

HeLa cells have proven to be suitable for the investigation since they are very easy to handle, they survive the process of transfection almost without damage and do not express HMGIC (the lack of HMGIC expression was proven by Northern blot). The cells are used for the transfection in plates with 6 wells.

For the process of transfection 2 μg DNA were mixed with cell medium (TC 199) without calf serum and antibiotics (both can interfere with the complex formation), final volume 100 μl.

10 μl Superfect is added to the DNA mixture. After mixing, it is incubated for 10 min at room temperature during which complexes form from the DNA and the SuperFect.

During the incubation the old medium is aspirated from the cells and the cells are rinsed with 1×PBS. The mixture of SuperFect and DNA is mixed with 800 μl cell medium containing 20% calf serum which is subsequently added to the cells. After an incubation (in an incubator at 37° C. and 6% CO₂) of 16-18 hours, fresh medium (20%) is added and it is incubated for a further 8-32 hours.

The experiment is evaluated using the “luciferase assay kit” of Stratagene (see below).

Luciferase Extraction and Determination of the Luciferase Concentration:

After incubation of the transfected cells, the medium is aspirated and 500 μl 1×cell lysis buffer is added. After incubating for 15 min at room temperature on a shaker, the cells lyse. The cell lysate is transferred to Eppendorf cups. This can be stored for a brief period at 4° C. It can be stored at −80° C. for a longer period but up to 50% luciferase activity is lost in this process.

In order to determine the luciferase concentration, 20 μl cell lysate is mixed with 100 μl luciferase assay reagent (LSA) (both should be at room temperature). The luciferin in the reaction mixture is converted with consumption of ATP and light quanta are generated.

luciferase substrate (luciferin)+ATP+O₂→light (560 nm)+oxyluciferin+AMP+PPᵢ

The emitted light quanta can be measured with a photocell of a luminometer. The determined values are stated in relative light units (RLU) and, as a ratio to other values, give information on the amount of luciferase formed.

Results:

Up to now two separate series of measurements have been carried out. Other measurements, in particular using promoter regions of BK and JVC viruses can be easily carried out in the present test system by cloning the appropriate promoter regions into the test vectors.

The results of the first 2 series of measurements are summarized in table 4

TABLE 4
Results of transfection experiments

| | | | | | 0.25 μg |
|---|---|---|---|---|---|
| | | | 1 μg H₃Hₓ | 0.5 μg H₃Hₓ | H₃Hₓ |
| | positive | negative | (1 μg | (1 μg | (1 μg |
| | control | control | pGL3-P) | pGL3-P) | pGL3-P) |
| relative light | 14,500 | 1,800 | 4,300 | 8,800 | 3,800 |
| units | | | | | |
| 1^{st} | | | | | |
| experiments | | | | | |
| relative light | 15,100 | 2,100 | 5,100 | 9,400 | 4,800 |
| units | | | | | |
| 2^{nd} | | | | | |
| experiment | | | | | |

The results of the measurements are higher than that of the negative control and under that of the positive control with a very strong promoter which clearly demonstrates a slight regulation of the viral promoter region by HMGIC.

This verifies the previously described involvement of viruses in the formation of leiomyomas, endometrial polyps and endometriosis.

### EXAMPLE 5

In this example the results of a PCR test are shown which was carried out in order to search for adenovirus-specific DNA sequences in myoma tissues. Specific oligonucleotides for all 6 subgenera of the adenoviruses are available to amplify viral DNA sequences.

Using the PureGene kit (Gentra Co. German supplier Biozym) DNA was isolated from 16 myomas, 6 cell cultures of myomas, 1 myometrium and 2 blood samples.

DNAs from the 8 myomas, all 6 cell cultures, the myometrium and the blood samples were used in a PCR. The following oligonucleotide pairs were used: HsgA1 (SEQ ID.No 1: aaggtgtcaatyatgtttg)/HsgA2 (SEQ ID.No 2: acggttacttkttt) and HsgB1 (SEQ ID.No.3: tctattccctacctggat)/HsgB2 (SEQ ID.No.4: actcttaacggcagtag) from the sequence of the hexon gene which amplify adenovirus DNA of group A and B respectively (Pring-Akerblom et al., J. Med. Virol. 58, 87-92, 99). The oligonucleotide pair HsgA amplifies a fragment of 299 bp and the oligonucleotide pair HsgB amplifies a fragment of 465 bp. The viral DNA samples from the adenovirus subgenera A (Ad18) and B (Ad7) were used as positive controls which were provided by Dr. Patricia Pring-Akerblom, “Medizinische Hochschule”, Hannover, “Institut für Virologie und Seuchenhygiene”, 30623 Hannover. The following PCR mixture was used.

500 ng DNA (myoma/blood) or 50 ng viral DNA

1.5 mM MgCl₂

0.5 μM of each primer

5 μl 10× PCR buffer without MgCl₂ (Sigma)

200 μM dNTP

2.5 U Taq polymerase (Sigma)

Each mixture contained a total volume of 50 μl. The following cycles were carried out:

| | | | |
|---|---|---|---|
| 1 x | 6 | min | 95° C. |
| 40 x | 40 | sec | 92° C. |
| | 30 | sec | 41° C. |
| | 40 | sec | 72° C. |
| 1 x | 5 | min | 72° C. |

The entire mixture was applied to a 1% agarose gel.

FIG. 6 shows the result of a PCR analysis to check for possible DNA sequences of adenoviruses with consensus primers of group B. With the exception of the viral control DNA, no amplification of the 299 bp fragment was detected using the primer pair HsgA. An amplification of the 465 bp fragment was detected using the primer pair HsgB in the case of four DNA fragments from uterine leiomyomas (My178.1; My174.3; My174.4; My161.7) (cell culture and primary tumour tissue) and the viral control DNA (human adenovirus 7). A corresponding product was not amplified either from blood DNA nor from DNA from normal myometrium from a uterine myomatosus (My187.6) or from the examined lung hamartomas (H). M5 denotes a marker (DNA standard V, Roche, Penzberg), arrow: position of the specific 465 bp fragment. Hence this unequivocally proves the presence of viral DNA sequences of the adenovirus type B in the myoma tissues.

### EXAMPLE 6

In order to characterize the PCR products in more detail these were cloned into a suitable vector and sequenced from the vector from both sides. On the basis of the results of the PCR analyses (see example 5) the DNA (6 fragments from 6 myoma DNA samples and I fragment from the virus control DNA Ad7) corresponding to the 465 bp amplificate was eluted from the agarose gel with the aid of the QIAEX II kit (Qiagen, Hilden, Germany) according to the manufacturer's instructions (QIAX II Handbook edition august 1996, p. 12-13). In addition a larger fragment was eluted which was amplified from the DNA My174.3 (cf example 5). In the following step the purified DNA was ligated into the vector pGEM-T Easy (Promega, Madison, USA) according to the manufacturer's instructions (Technical Manual pGEM-T and pGEM-T Easy Vector Systems, p. 11) and the vector construct was cloned into E. coli (Technical Manual pGEM-T and pGEM-T Easy Vector Systems, p. 12-13). Plasmid DNA of positive bacterial clones was isolated with the aid of the QIAprep kit (Qiagen, Hilden, Germany) according to the manufacturer's instructions (QIAprep Miniprep Handbook edition April 1998, p. 18-19). The cloned inserts were sequenced using the oligonucleotides M13 universal and M 13 reverse and with the aid of an automated sequencing unit (373 Applied Biosystems, Weiterstadt, Germany). Comparative analyses of the sequences were carried out with the aid of the data banks published on the internet (access: http://www.ncbi.nlm.nih.gov/) and search methods (Advanced BLAST, data bank “nr” without stating a certain species) of the American National Center for Biotechnology (NCBI) information of the National Institute of Health.

Results: The amplified sequences from all tumours and tumour cell cultures correspond (with some deviations see FIG. 3, FIG. 4 and table 5) to the 465 bp PCR fragment of the positive control. The sequence which was obtained in the analysis of the large fragment of the amplificate from My174.4 did not yield a match to a viral sequence in the comparative analysis.

TABLE 5

| | best match | number of | characteristic |
|---|---|---|---|
| PCR product | accession number | mutations | features |
| M 3-3 (control AD7) | AF065065 | 2 | 4xN, 2 exchange |
| | | | mutations |
| M 2-3 (myoma DNA) | X765551 | 0 | |
| M 5-1 (myoma DNA) | AF065065 | 0 | |
| M 6-1 (myoma DNA) | AF065068 | 1 | no exchange |
| | | | mutation |
| M 7-1 (myoma DNA) | X765551 | 8 | no exchange |
| | | | mutation |
| M 8-2 (myoma DNA) | X765551 | 10 | 1 exchange |
| | | | mutation |
| M 9-2 (myoma DNA) | AF065065 | 8 | no exchange |
| | | | mutation |

Hence this shows that the viral sequences which were amplified from the individual myomas have the greatest similarity to the sequence of the adenovirus type 7 and can differ from one another as a result of point mutations.

Explanation of FIG. 3a-c, FIG. 4a-b and Table 5

The comparative analysis of the DNA and protein sequences gave evidence for a high homology of the sequences amplified from the myoma tissues to published sequences from the hexon region of the adenovirus type 7. The individual DNA sequences have point mutations and differ from one another. With the exception of a point mutation which leads to an amino acid substitution in the protein sequence M 8-2, all other mutations prove to be so-called silent mutations. The differences in the sequences are labelled by boxes in FIGS. 3a-c. The nomenclature used: X765551, AF 065068, AF 65065: published sequences of adenoviral hexon genes, M 3-3P: positive control (see example 5), M 2-3, M 5-1, M 6-1, M 7-1, M 8-2, M 9-2: different sequences of amplificates of myoma tissue.

FIGS. 4a-b show a comparative analysis of all DNA sequences obtained from the various myoma tissues. The differences are labelled by boxes. The nomenclature corresponds to that of FIG. 3a-c.

### EXAMPLE 7

HMGI(Y) proteins can influence the expression of viral proteins by binding to viral promoters. The published sequence of the promoter of the gene E1a (accession number X03000 or NCBI data bank; E1a is an adenoviral gene the gene product of which has been ascribed a transforming function) and the published data on the binding modality of HMGI(Y) to DNA sequences (cf. Yic et al., Molecular and Cellular Biology 17: 3649-3662, 1997) were used as a basis to examine whether HMGI(Y) can bind to the promoter sequence. Proteins of the HMGI-Y group contain three binding domains 2 of which can bind in parallel to DNA sequences that are composed of a sequence of at least 4 adenines and thymidines. These DNA sequences are ideally 10 or 20 base pairs apart since the binding HMGI-Y proteins can span 1 to 2 helical coils of the DNA due to the position of the binding domains (Yie et al., Molecular and Cellular Biology, 17: 3649-3662, 1997). When HMGI-Y proteins bind in the described manner to cellular or viral promoters, the promoter-mediated action is modified in the sense of an activation or inhibition. The promoter sequence of the adenoviral protein E1A (accession number X03000, nucleotides 1-511, source: AD7) was identified by means of a sequence comparison using the data banks published by the NCBI (see example 6). Numerous binding sites for HMGIY proteins were determined on the basis of the described criteria (cf. FIG. 5). As an example two of these identified binding sites which can be bound concurrently by two binding domains of a HMGI-Y protein have been linked by a bracket.

Hence this demonstrates that HMGI-Y proteins can bind to the promoter sequence.

### EXAMPLE 8

On the basis of the sequence of the left end of AD7 (accession number X03000) published by the NCBI, the oligonucleotides ADE1Bg12S: (SEQ ID.NO.5): gaa gat ctt tat aga tgg aat ggt gcc aac at and ADE1Hi3AS (SEQ ID NO.6): ccc aag ctt aaa act ctt ctc gct ggc agt c were selected which can bind to the promoter sequence of the E1A gene of the adenovirus 7. The oligonucleotide ADE1Bg12S contains a Bg1 II cleavage site and the oligonucleotide ADE-Hi3AS contains a HindlIl cleavage site. Both cleavage sites are underlined in the sequence shown above. The oligonucleotides were used to amplify a 521 bp fragment from the AD7 promoter region and this was cloned (pAD7PROM) by means of standard methods (Maniatis) into the luciferase reporter vector pGL3 enhancer (Promega) by means of the cleavage sites Bg1I and HindIII. The AD7-DNA which had already served as a positive control in example 5 was used as a template. The amplified fragment then acts as a promoter for the firefly luciferase gene which is present in the vector and the activity of which can be measured by a luciferase assay (Dual-Luciferase-Reporter-Assay-System, Promega). The co-transfection of the above-mentioned construct with an HMGIC expression vector (H3HX) in HeLa cells as well as in primary myometrial cells from the 1^{st} passage of the tissue culture demonstrated a modification of the E1A protein function by HMGIC. All transfections were carried out using SuperFect according to the protocol of the Qiagen Company. The original protocol was modified such that 1 μg of the respective constructs was used in each case and the cells were not washed with PBS. The incubation was increased from a few hours to an overnight incubation and after this the SuperFect was not removed but instead 3 ml medium was added to the cultures. This showed that the expression of the transformed adenoviral protein E1A is influenced by the binding of HMGIC proteins in the viral promoter region. Two additional co-transfections were carried out as negative controls in one of which the expression vector contained no cloned HMGIC sequence and additionally the transfection was carried out without the addition of the HMGIC expression construct. The pGL3 control vector which contains an SV40 promoter and SV40 enhancer served as a positive control. In order to exclude inaccuracies in the cell culture such as different numbers of cells per cell culture dish, differences in the efficiency of the transfection and cell lysis, the activity of the experimental reporter construct (see above) was normalised by co-transfection with an internal pRL control vector (pRL-TK, Renilla luciferase). The luciferase measurements were carried out according to the protocol of the dual-luciferase-reporter-assay system from Promega.

### EXAMPLE 9

A further example for the induction of tissue changes by infection of the tissue cells with adenoviruses are lung hamartomas. This example describes the strategy for examining and for detecting adenoviral genomes in various lung hamartoma tissues.

The DNA was Isolated from 10 Cell Cultures of Hamartomas using the PureGene Kit (Gentra Co, German Supplier Biozym).

DNAs from 7 cell cultures were used in a PCR. The following oligonucleotide pairs were used HsgA1/HsgA2, HsgB1/HsgB2, HsgC1 [(SEQ ID NO.7): acctttgactcttctgt)]/HsgC2 [(SEQ ID NO.8): tccttgtatttagtatc], HsgD1 [(SEQ ID NO.9): ccatcatgttcgactcct]/HsgD2 [(SEQ ID NO.10): aggtagccggtgaagcc], HsgE1 [(SEQ ID NO.11): gactcttccgtcagctgg]/HsgE2 [(SEQ ID NO.12): gctggtaacggcgctct] and HsgF1 [(SEQ ID NO.13): atttctattccttcgcg]/HsgF2 [(SEQ ID NO.14): tcaggcttggtacggcc] from the sequence of the hexon gene which amplify adenovirus DNA of groups A to F respectively (Pring-Akerblom et al., J. Med. Virol. 58, 87-92, 99). The oligonucleotide pair HsgA amplifies a fragment of 299 bp, the oligonucleotide pair HsgC amplified a fragment of 269 bp, the oligonucleotide pair HsgD amplifies a fragment of 331 bp, the oligonucleotide pair HsgE amplifies a fragment of 399 bp and the oligonucleotide pair HsgF amplifies a fragment of 586 bp. The viral DNA samples used as a control were also provided by Dr. Pring-Akerblom (cf. example 5: subgenus A: Ad18, subgenus B: Ad7, subgenus C: Ad1, subgenus D: Ad17; subgenus E: Ad4; subgenus F: Ad41).

The following PCR mixture was used:

500 ng DNA (tissue, cell culture) or 50 ng viral DNA

1.5 mM MgCl₂

0.5 μM of each primer

5 μl 10× PCR buffer without MgCl₂ (Sigma)

200 μM dNTP

2.5 U Taq polymerase (Sigma)

Each mixture contained a total volume of 50 μl. The following cycles were carried out.

| | | | |
|---|---|---|---|
| 1 x | 6 | min | 95° C. |
| 40 x | 40 | sec | 92° C. |
| | 30 | sec | 41° C. |
| | 40 | sec | 72° C. |
| 1 x | 5 | min | 72° C. |

The entire mixture was applied to a 1.5% agarose gel. No fragment of the respective expected length is amplified from the various lung hamartoma DNA samples using the oligonucleotide pairs HsgA1/HsgA2, HsgB1/HsgB2, HsgC1/HsgC2 and HsgF1/HsgF2 although the corresponding fragment from the viral control DNA was amplified. A 331 bp fragment was amplified from the DNA samples of three lung hamartomas using the oligonucleotides HsgD1/HsgD2. A 399 bp product was amplified from 4 additional lung hamartoma DNA samples.

This clearly shows that an infection of the original cell with adenoviruses of group D or E leads to the formation of lung hamartomas.

### EXAMPLE 10

In order to examine whether cells of lung hamartomas can be permissive in vitro, HeLa cells were co-cultured with cells of a lung hamartoma. HeLa cells are often used to multiply adenoviruses and exhibit cytopathological effects after infection.

The hamartoma tissue that was used was derived from a tumour having a chromosomal translocation t(6;14)(p21;q24). After the operation it was stored for 26 hours in Hank's solution at room temperature and then cut up into ca. 1 mm³ cubes using scissors and a scalpel. Then it was subjected to a further enzymatic disintegration using collagenase by routine methods (Kazmierczak et al., Oncogene, 12: 515-521). The resulting cell suspension was divided up and placed in four 25 cm² cell culture flasks which were each prefilled with 5 ml cell culture medium (medium 199 containing 20% foetal calf serum and antibiotics). After a three day culture period at 3° C., 5% CO₂, a confluent monolayer had formed in the flasks. The cells were then detached with a trypsin/EDTA solution using standard methods and distributed into two new culture flasks. After two hours ca. 3×10⁵ HeLa cells in 1 ml culture medium were added to each of the culture flasks. After a one day culture period the flasks contained about 50% HeLa cells and fibroblast-like hamartoma cells. The serum concentration in the medium was reduced to 10%. After two further days the HeLa cells exhibited changes in cell morphology in several positions and ellipsoidal cells began to detach themselves from the bottom of the culture vessels. After four days only a few groups of HeLa cells were detectable which, however, proliferated.

The cytopathological effect which occurs may be due to the hamartoma cells that are permissive for adenoviruses that have infected the Hela cells.

### EXAMPLE 11

Assuming that an activation of genes of the HMGI(Y) family or an increased expression thereof leads to an increased growth of adenovirus-transformed cells, the following experiment was carried out for clarification.

A hamster cell line which has a single integrate of the human adenovirus in the genome was used as a recipient for the transfection experiment. The HMGIC gene was transiently overexpressed in the cells by means of an expression vector for the wildtype HMGIC as described in example 4. Cells transfected with an empty vector served as a negative control. The cell counts were determined in the preparations 12 hours, 24 hours and 36 hours after transfection. Whereas there were no clear differences between the cultures after 12 hours, the cultures in which the HMGIC was overexpressed had on average 1.2-fold or 1.47-fold increased cell counts after 24 and 36 hours respectively compared to the control cultures. Hence these transfection experiments show the growth advantage that is mediated by overexpression of HMGIC in adenovirus-transforrned cells and thus confirms the above opinion that adenoviral transformed cells exhibit increased growth when genes of the HMGI(Y) family are activated.

### EXAMPLE 12

In order to examine endometriosis foci for the presence of adenovirus group B which were also detected in uterine leiomyomas, tissue samples of macroscopically identified endometriosis foci from 4 female patients were frozen in liquid nitrogen immediately after the operation. The DNA was then isolated from the frozen samples by conventional methods. This DNA was used for the PCR analysis described in example 5. An amplification of the 465 bp fragment which is typical for adenoviruses of group B was found in two of the analysed samples. The results of a serial dilution in which viral control DNA was mixed with myometrium in which no amplification was detectable, showed that the method cannot detect more than on average 2 viral genomes per host cell. Hence the results of the analysis shown here shows that in the case of the two positive samples more than 2 viral genomes must have been present per cell.

### EXAMPLE 13

In order to examine endometrial polyps for the presence of adenovirus of group B which were also detected in uterine leiomyomas, tissue samples of macroscopically identified endometrial polyps from 3 female patients were frozen in liquid nitrogen immediately after the operation. The DNA was then isolated from the frozen samples by conventional methods. This DNA was used for the PCR analysis described in example 5. An amplification of the 465 bp fragment which is typical for adenoviruses of group B was found in one of the analysed samples. The results of a serial dilution in which viral control DNA was mixed with myometrium in which no amplification was detectable, showed that the method cannot detect more than on average 2 viral genomes per host cell. Hence the results of the analysis shown here shows that in the case of the positive sample more than 2 viral genomes must have been present per cell.

The features of the invention disclosed in the previous description, the claims and the figures can be important individually as well as in any combination for the realization of the invention in its various embodiments.

1. (canceled)

2. (canceled)

3. (canceled)

4. (canceled)

5. Canceled).

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. Use of a preparation which contains an antiviral agent to produce a medicament for preventing and/or treating tissue changes, wherein
the tissue change involves tissue of mesenchymal origin or tissue changes derived therefrom and
at least one cell of the tissue constituting the tissue change is infected with a virus.

49. Use of a preparation comprising an antiviral agent to produce a medicament for preventing and/or treating a tissue changes, wherein the tissue change involves tissue of mesenchymal origin or tissue changes derived therefrom and in particular a tissue change selected from the group comprising leiomyomas, in particular leiomyomas of the uterus, endometrial polyps, endometriosis, fibroadenomas, in particular fibroadenomas of the mamma, phyllodes tumours, in particular of the mamma, hamartomas, in particular of the mamma, prostate adenomas, lipomas, angiomysomas, enchondromas, pleomorphic adenomas, especially of the salivary glands of the head, colon polyps, especially colon adenomas, atheromas and carcinomas that develop therefrom.

50. Use as claimed in 48, characterized in that the preparation is directed against the virus.

51. Use as claimed in one of the claims 48, 49, or 50, characterized in that the preparation is effective against a virus the nucleic acid of which contains at least one binding site for a gene product of genes of the HMGI(Y) family or derivatives thereof.

52. Use as claimed in one of the claims 48, 49, or 50, characterized in that the preparation is effective against a virus the nucleic acid of which codes for a gene product and this gene product interacts with at least one gene product of genes of the HMGI(Y) family or derivatives thereof.

53. Use as claimed in claim 4, characterized in that the binding site on the nucleic acid of the virus has the characteristic structural and sequence features of a first AT-rich sequence.

54. Use as claimed in claim 53, characterized in that the binding site on the nucleic acid of the virus, in addition to the first sequence, also has the following characteristic structural and sequence features:
a second AT-rich sequence is present and
the first and second sequence are arranged at a spatial distance from one another.

55. Use as claimed in claim 54, characterized in that the spatial distance is selected such that the first sequence and the second sequence are arranged relative to one another in one plane on the nucleic acid.

56. Use as claimed in one of the claims 48, 49, 50, 51, 52, 53, 54 or 55, characterized in that the genes of the HMGI(Y) family comprise MAG genes, HMGIC, HMGIY, aberrant transcripts of genes of the HMGI(Y) family and derivatives thereof.

57. Use as claimed in one of the claims 48, 49, 50, 51, 52, 53, 54, 55 or 56, characterized in that the virus infecting the at least one cell of the tissue constituting the tissue change is one as claimed in one of the previous claims.

58. Use as claimed in one of the claims 48, 49, 50, 51, 52, 53, 54, 56 or 57, characterized in that the preparation is effective against a virus from the group of DNA viruses and in particular adenoviruses and/or herpes viruses.

59. Use as claimed in one of the claims 48, 49, 50, 51, 52, 53, 54, 56, 57 or 58, characterized in that the tissue of mesenchymal origin is at least partially infected with a virus from the group of DNA viruses and in particular adenoviruses and/or herpes viruses.

60. Use as claimed in one of the claims 48, 49, 50, 51, 52, 53, 54, 56, 57, 58 or 59, characterized in that the tissue change comprises a proliferation of at least one mesenchymal cell which is infected with a virus as claimed in one of the previous claims.

61. Use as claimed in claim 60, characterized in that the proliferation is a clonal proliferation.

62. Use as claimed in one of the claims 48, 49, 50, 51, 52, 53, 54, 55, 56, 57, 58, 59, 60 or 61, characterized in that the tissue change comprises an epithelial component.

63. Use as claimed in claim 60, characterized in that the epithelial component has at least one cell which is infected with a virus as claimed in one of the previous claims.

64. Use as claimed in one of the claims 48, 49, 50, 51, 52, 53, 54, 55, 56, 57, 58, 59, 60, 61, 62 or 63, characterized in that the cell infected with the virus has a chromosomal change.

65. Use as claimed in claim 64, characterized in that the chromosomal change affects at least one HMGI(Y) gene of the infected cell.

66. Use as claimed in claim 65, characterized in that the HMGI(Y) gene is selected from the group comprising MAG genes, HMGIC, HMGIY, aberrant transcripts of genes of the HMGI(Y) family and derivatives thereof.

67. Use as claimed in one of the claims 48 and 50, 51, 52, 53, 54, 55, 56, 57, 58, 59, 60, 61, 62, 63, 64, 65 or 66, characterized in that the tissue change is selected from the group comprising leiomyomas, in particular leiomyomas of the uterus, endometiral polyps, endocmetriosis, fibroademas, in particular fibroadenomas of the mamma, phyllodes tumour, in particular of the mamma, hamartomas, in particular of the mamma and the lung, prostate adenomas lipomas, aggressive angiomyxomas, enchondromas, pleomorphic adenomas, especially of the salivary glands of the head, colon polyps, especially colon adenomas, atheromas and carcinomas that develop therefrom.

68. Use as claimed in claim 67, characterized in that the carcinomas that have formed are selected from the group comprising colon carcinomas and prostate carcinomas.

69. Use as claimed in one of the claims 48, 49, 50, 51, 52, 53, 54, 55, 56, 57, 58, 59, 60, 61, 62, 63, 64, 65, 66, 67 or 68, characterized in that the agent is selected from the group comprising vaccines, antibodies, preparations that inhibit the replication, transcription or translation of viral genes in particular of genes of adenovirus and/or herpes viruses; preparations that recognize and/or destroy cells infected by viruses especially adenoviruses and/or herpes viruses and preparations which achieve an antiviral effect by their effector-cell-stimulating action.

70. Use as claimed in claim 69, characterized in that the vaccine contains an antibody which is directed against the virus as claimed in one of the previous claims or a part thereof.

71. Use as claimed in claim 69, characterized in that the vaccine contains a particle of the virus as claimed in one of the previous claims or a part thereof.

72. Use as claimed in claim 70, characterized in that the antibody is selected from the group comprising monoclonal antibodies, polyclonal antibodies, polyvalent antibodies, antibody fragments and derivatives thereof.

73. Use as claimed in one of the claims 69, 70, 71 or 72, characterized in that the medicament for immunization is suitable for immunizing against viruses that are associated with the pathogenesis and/or aetiology of tissue changes as claimed in one of the previous claims.

74. Use of a method to determine an antiviral agent to produce a preparation for preventing and/or treating tissue changes as claimed in one of the previous claims and/or determining viruses against which the preparation as claimed in one of the previous claims is directed, which comprises the steps:
a) transfecting a cell culture having a normal karyotype which is derived from a tissue that contains the tissue change as claimed in one of the previous claims, with an expression vector for a gene of the HMGI(Y) family or a derivative thereof,
b) comparing the RNA pattern of the transfected cells with that of control cultures, and
c) examining RNA(s) that are expressed or expressed more strongly in the transfected cultures compared to the control cultures for the presence of viral elements by sequence homology.

75. Use of a method comprising carrying out a PCR test in which the primer (pairs) used for the PCR conform to the sequence of viral nucleic acids in order to determine viruses,
which are suitable for producing a medicament as claimed in one of the previous claims and/or
against which the preparation as claimed in one of the previous claims is directed.

76. Use of a method comprising:
a) setting up a cDNA library of a tissue which contains the tissue change as claimed in one of the previous claims in which a gene of the HMGI(Y) family or a derivative thereof is activated or can be activated and
b) screening the cDNA library with a virus-specific probe or
c) analyzing the cDNA clones for viral sequences or
d) comparing with a cDNA library from a normal reference tissue to determine viruses that are suitable for producing a medicament as claimed in one of the previous claims and/or against which the preparation as claimed in one of the previous claims is directed.

77. Use of a method as claimed in one of the claims 74, 75 or 76, characterized in that the gene of the HMGI(Y) family is selected from the group comprising HMGIC, HMGIY, MAG, aberrant transcripts of genes of the HMGI(Y) family and derivatives thereof.

78. Use of a method as claimed in one of the claims 74, 75 or 76, characterized in that the virus, the viral element or the virus-specific probe is selected from the group of viruses which comprises the viruses as claimed in one of the previous claims.

79. Use of a method as claimed in one of the claims 74, 75, 76, 77, or 78 to determine viruses against which it is possible to immunize in order to prevent and/or treat tissue changes as claimed in one of the previous claims.

80. Use of a device for determining a virus involved in the pathogenesis of tissue changes as claimed in one of the previous claims which comprises a gene product of genes of the HMGI(Y) family or a part thereof or derivatives thereof bound to a carrier.

81. Use of a device as claimed in claim 80, characterized in that the viral nucleic acid in addition to the first sequence, also has the following characteristic structural and sequence features:
a second AT-rich sequence is present and
the first sequence and second sequence are arranged at a spatial distance from one another.

82. Use of a device as claimed in claim 81, characterized in that the spatial distance is selected such that the first sequence and the second sequence are arranged relative to one another in one plane on the nucleic acid.

83. Use of a diagnostic method, characterized in that a body fluid from a patient that may have such a tissue change is examined for the presence of antibodies against viruses as described in one of the previous claims, preferably DNA viruses and especially preferably adenoviruses and/or herpes viruses, to diagnose a tissue change in which the tissue change comprises a tissue change as claimed in one of the previous claims.

84. Use of a diagnostic method, characterized in that a body fluid from a patient that may have such a tissue change is examined for the presence of antigens of viruses as described in one of the previous claims, preferably DNA viruses and especially preferably adenoviruses and/or herpes viruses, to diagnose a tissue change in which the tissue change comprises a tissue change as claimed in one of the previous claims.

85. Use of a diagnostic method, characterized in that a tissue sample is reacted with a preparation which is selected from the group comprising antibodies which react with viruses as described in one of the previous claims preferably DNA viruses and especially preferably adenoviruses and/or herpes viruses or parts thereof, antigens that are derived from viruses as described in one of the previous claims, preferably DNA viruses and especially preferably adenoviruses and/or herpes viruses, to diagnose a tissue change, characterized in that:
a) the tissue change comprises one as claimed in one of the previous claims, and
b) if viruses as described in one of the previous claims and preferably DNA viruses and especially preferably adenoviruses and/or herpes viruses are present, a complex is formed form the preparation and the virus and
c) the complex is detected.
